(19) United States
(12) Patent Application Publication
(10) Pub. No.: US 2013/0115173 A1
(43) Pub. Date: May 9, 2013

(54) Stable Dermatological Aerosol Foams Utilizing Reactive Propellants

(71) Applicant: Precision Dermatology, Inc., Cumberland, RI (US)

(72) Inventors: Mark W. Trumbore, Westford, MA (US); Pinaki Ranjan Majhi, Sharon, MA (US)

(73) Assignee: Precision Dermatology, Inc., Cumberland, RI (US)

(21) Appl. No.: 13/667,209

(22) Filed: Nov. 2, 2012

Related U.S. Application Data
(60) Provisional application No. 61/555,045, filed on Nov. 3, 2011.

(51) Int. Cl. A61K 9/12

(52) U.S. Cl. CPC A61K 9/124
USPC 424/45

(57) ABSTRACT

Described herein are chemically-stabile formulations comprising a reactive propellant. The formulations are delivered as an aerosol foam, and are suitable for topical application. The aerosol propellant may be a hydrofluoroolefin propellant. The emulsions or compositions may comprise reactive active agents, such as peroxides, retinoids, or quinones. Also described are methods of treating skin disorders, comprising the step of applying to an affected area of a subject in need thereof a therapeutically-effective amount of an inventive emulsion or aerosol composition.

### RELATED APPLICATIONS

This application claims the benefit of priority to U.S. Provisional Patent Application Ser. No. 61/555,045, filed Nov. 3, 2011, the contents of which are hereby incorporated by reference.

### BACKGROUND

There are several possible choices of propellants for use in aerosol foam formulations of reactive active pharmaceutical ingredients, including, but not limited to, CFCs, hydrocarbons, compressed gases, and HFAs. The Montreal Protocol has banned the use of chlorofluorocarbons (CFCs) due to their ability to deplete the ozone layer. Montreal Protocol on Substances that Deplete the Ozone Layer, United Nations Environmental Programme, 1987. Alternatively, hydrocarbon propellants demonstrate very low reactivity and good resistance to free-radical attack. However, hydrocarbon propellants are highly flammable. Compressed inert gases, such as nitrogen and carbon dioxide, may be used as an aerosol propellant. While offering good chemical stability due to their non-reactivity, they are unable to deliver consistent product delivery throughout the life of the aerosol can due to their high vapor pressures. Hydrofluoroalkane propellants (HFAs, also known as hydrofluorocarbons, HFCs) are also an option. These propellants are pharmaceutically acceptable, generally non-reactive, and ozone-friendly. Unfortunately, HFA propellants exhibit high global warming potential, which is leading to regulatory limits on their use. Recently, a new class of aerosol propellants has been developed as a replacement for HFAs. These propellants consist of volatile hydrofluoroolefins (HFOs) either in combination or singly. HFOs have a reactive double bond in their structure. When released into the atmosphere, the propellant double bond is attacked, leading to propellant decomposition and subsequent rapid clearing from the atmosphere.

Many classes of pharmaceutically active ingredients are well known to be highly reactive, examples of these include peroxides (such as benzoyl peroxide), retinoids (such as Tretinoin and retinol), vitamins (such as vitamin E (tocopherol)), and quinones (such as hydroquinone). When developing products containing these ingredients great care is taken to assure that the inactive ingredients chosen for the formulation do not react with the active ingredients. Likewise, great care is taken in the choice of packaging to assure that the active ingredients do not react with either the packaging materials or the outside environment.

Aerosol packaging systems appear to be well suited for use in the delivery of reactive active ingredients due to their impermeability to the outside environment and the presence of a wide variety of low reactivity interior coatings. However, these systems suffer from the fact that they are pressurized. As is well known, variations in pressure, similar to increases in temperature, can have dramatic effects on reaction kinetics, greatly increasing the rate of chemical reaction. This inherent increase in reactivity makes the combination of reactive active ingredients and reactive aerosol propellants an unappealing one.

### SUMMARY OF THE INVENTION

In certain embodiments, the invention relates to an emulsion, comprising:

water,

an emulsifier or surfactant,

a moisturizer or emollient,

an antioxidant or preservative, and

an active agent.

In certain embodiments, the invention relates to an emulsion, comprising:

water,

an emulsifier or surfactant,

a moisturizer or emollient,

an antioxidant or preservative,

a pH adjuster, and

an active agent.

In certain embodiments, the invention relates to an emulsion, comprising:

water,

an emulsifier or surfactant,

a moisturizer or emollient,

an antioxidant or preservative,

a pH adjuster,

a buffer, and

an active agent.

In certain embodiments, the invention relates to a composition, comprising:

any one of the aforementioned emulsions; and

a propellant.

In certain embodiments, the invention relates to a composition, comprising:

any one of the aforementioned emulsions;

a propellant; and

a purge gas.

In certain embodiments, the invention relates to any one of the aforementioned compositions, wherein the propellant is selected from the group consisting of Z-1,2,3,3,3-pentafluoropropene (HFO-1225yeZ), E-1,2,3,3,3-pentafluoropropene (HFO-1225yeE), 1,1,3,3,3-pentafluoropropene (HFO-1225zc), 1,1,2,3,3-pentafluoropropene (HFO-1225yc), 2,3,3,3-tetrafluoropropene (HFO-1234yf), trans-1,3,3,3-tetrafluoroprop-1-ene (HFO-1234zeE or HBA-1), cis-1,3,3,3-tetrafluoropropene (HFO-1234zeZ), and 3,3,3-trifluoropropene (HFO-1243zf), and combinations/mixtures thereof.

In certain embodiments, the invention relates to any one of the aforementioned emulsions or compositions, wherein the active agent is reactive.

In certain embodiments, the invention relates to a method of treating a skin disorder, comprising the step of applying to an affected area of a subject in need thereof a therapeutically-effective amount of any one of the aforementioned emulsions or compositions.

### BRIEF DESCRIPTION OF THE FIGURES

FIG. 1 tabulates the constituents (and their relative quantities) of various embodiments of the emulsions of the invention.

FIG. 2 tabulates as a function of time the benzoyl peroxide concentrations in a number of formulations of the invention comprising HFO aerosol propellants.

### DETAILED DESCRIPTION OF THE INVENTION

### Overview

In certain embodiments, the invention relates to formulations comprising an emulsion and a propellant. In certain embodiments, the compositions do not contain volatile lower alcohols (e.g., ethanol). In certain embodiments, the compositions comprise an aerosol propellant. In certain embodiments, the aerosol propellant is a hydrofluoroolefin (HFO) propellant.

In certain embodiments, the invention relates to an aerosol foam compositions or formulations suitable for dermatological use and containing reactive active ingredients which are chemically stable in the presence of double bond containing HFO aerosol propellants. In certain embodiments, the aerosol propellant is dissolved or emulsified in the formulation so that it is in intimate contact with all components of the formulation. In certain embodiments, the formulations comprise oil-in-water emulsions with the reactive active ingredients incorporated in either the oil phase or the water phase.

In certain embodiments, the compositions produce a foam upon actuation of an aerosol container charged with the composition. In certain embodiments, the compositions immediately produce a foam upon actuation of an aerosol container charged with the composition. In certain embodiments, the foams are stable against collapse. In certain embodiments, the foams are both time- and temperature-stable. In certain embodiments, the foam is moisturizing. In certain embodiments, the foam is non-irritating. In certain embodiments, the dispensed foam has a density between about 0.05 and about 0.5 g/cm³. In certain embodiments, the foam rubs-in quickly without a greasy residue. In certain embodiments, the dispensed foam is easily spread over large body surface areas. In certain embodiments, the foam rapidly collapses when subjected to shear forces, allowing for quick and efficient application to large body surface areas. In certain embodiments, the dispensed foam is compatible with a wide range of active pharmaceutical ingredients, including reactive or unstable pharmaceutical ingredients. In certain embodiments, the dispensed foam is suitable for the topical treatment of skin disorders.

In certain embodiments, the dispensed foam is as effective for the treatment of skin disorders as currently commercially-available prescription creams, lotions, and ointments.

### DEFINITIONS

For convenience, certain terms employed in the specification and appended claims are collected here. These definitions should be read in light of the entire disclosure and understood as by a person of skill in the art.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

The phrase “or,” as used herein in the specification and in the claims, should be understood to mean “either or both” of the elements so conjoined, i.e., elements that are conjunctively present in some cases and disjunctively present in other cases. Multiple elements listed with “or” should be construed in the same fashion, i.e., “one or more” of the elements so conjoined. Other elements may optionally be present other than the elements specifically identified by the “or” clause, whether related or unrelated to those elements specifically identified. Thus, as a non-limiting example, a reference to “A or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### Exemplary Constituents of Emulsions and Compositions of the Invention

Exemplary identities of various constituents of the compositions of the present invention are described below.

### 1. Propellants

In one embodiment, the propellant is a HFO or a mixture of one or more hydrofluoroolefins. Suitable hydrofluoroolefins include, but are not limited to, Z-1,2,3,3,3-pentafluoropropene (HFO-1225yeZ), E-1,2,3,3,3-pentafluoropropene (HFO-1225yeE), 1,1,3,3,3-pentafluoropropene (HFO-1225zc), 1,1,2,3,3-pentafluoropropene (HFO-1225yc), 2,3,3,3-tetrafluoropropene (HFO-1234yf), trans-1,3,3,3-tetrafluoroprop-1-ene (HFO-1234zeE or HBA-1), cis-1,3,3,3-tetrafluoropropene (HFO-1234zeZ), and 3,3,3-trifluoropropene (HFO-1243zf), and mixtures and admixtures of these and other HFOs that are currently approved or may become approved for medical use are suitable. Hydrocarbon, chlorofluorocarbon (CFC), or hydrofluoroalkane (HFA) propellants can also be used in the present invention.

### 2. Vehicles

Suitable topical vehicles and vehicle components for use with the formulations of the invention are well known in the cosmetic and pharmaceutical arts, and include such vehicles (or vehicle components) as water; organic solvents such as alcohols (particularly lower alcohols readily capable of evaporating from the skin such as ethanol), glycols (such as propylene glycol, butylene glycol, and glycerol (glycerin)), aliphatic alcohols (such as lanolin); mixtures of water and organic solvents (such as water and alcohol), and mixtures of organic solvents such as alcohol and glycerol (optionally also with water); lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile) such as cyclomethicone, dimethiconol, dimethicone, and dimethicone copolyol; hydrocarbon-based materials such as petrolatum and squalane; and other vehicles and vehicle components that are suitable for administration to the skin, as well as mixtures of topical vehicle components as identified above or otherwise known to the art.

In one embodiment, the compositions of the present invention are oil-in-water emulsions. Liquids suitable for use in formulating compositions of the present invention include water, and water-miscible solvents such as glycols (e.g., ethylene glycol, butylene glycol, isoprene glycol, propylene glycol), glycerol, liquid polyols, dimethyl sulfoxide, and isopropyl alcohol. One or more aqueous vehicles may be present.

In one embodiment, formulations without methanol, ethanol, propanols, or butanols are desirable.

### 3. Surfactants and Emulsifiers

Many topical formulations contain chemical emulsions which use surface active ingredients (emulsifiers and surfactants) to disperse dissimilar chemicals in a particular solvent system. For example, most lipid-like (oily or fatty) or lipophilic ingredients do not uniformly disperse in aqueous solvents unless they are first combined with emulsifiers, which form microscopic aqueous soluble structures (droplets) that contain a lipophilic interior and a hydrophilic exterior, resulting in an oil-in-water emulsion. In order to be soluble in aqueous media, a molecule must be polar or charged so as to favorably interact with water molecules, which are also polar. Similarly, to dissolve an aqueous-soluble polar or charged ingredient in a largely lipid or oil-based solvent, an emulsifier is typically used which forms stable structures that contain the hydrophilic components in the interior of the structure while the exterior is lipophilic so that it can dissolve in the lipophilic solvent to form a water-in-oil emulsion. It is well known that such emulsions can be destabilized by the addition of salts or other charged ingredients which can interact with the polar or charged portions of the emulsifier within an emulsion droplet. Emulsion destabilization results in the aqueous and lipophilic ingredients separating into two layers, potentially destroying the commercial value of a topical product.

Surfactants suitable for use in the present invention may be ionic or non-ionic. These include, but are not limited to: polysorbates (Polysorbate 20, Polysorbate 40, Polysorbate 60, Polysorbate 80), steareth-10 (Brij 76), sodium dodecyl sulfate (sodium lauryl sulfate), lauryl dimethyl amine oxide, cetyltrimethylammonium bromide (CTAB), polyethoxylated alcohols, polyoxyethylene sorbitan, octoxynol, N,N-dimethyldodecylamine-N-oxide, hexadecyltrimethylammonium bromide (HTAB), polyoxyl 10 lauryl ether, bile salts (such as sodium deoxycholate or sodium cholate), polyoxyl castor oil, nonylphenol ethoxylate, cyclodextrins, lecithin, dimethicone copolyol, lauramide DEA, cocamide DEA, cocamide MEA, oleyl betaine, cocamidopropyl betaine, cocamidopropyl phosphatidyl PG-dimonium chloride, dicetyl phosphate (dihexadecyl phosphate), ceteareth-10 phosphate, methylbenzethonium chloride, dicetyl phosphate, ceteth-10 phosphate (ceteth-10 is the polyethylene glycol ether of cetyl alcohol where n has an average value of 10; ceteth-10 phosphate is a mixture of phosphoric acid esters of ceteth-10), Brij S10 (polyethylene glycol octadecyl ether, average Mₙ˜711), and Poloxamers (including, but not limited to, Poloxamer 188 (HO(C₂H₄O)ₐ(CH(CH₃)CH₂O)_{b}(C₂H₄O)ₐH, average molecular weight 8400) and Poloxamer 407 (HO(C₂H₄O)ₐ(CH(CH₃)CH₂O)_{b}(C₂H₄O)ₐH, wherein a is about 101 and b is about 56)). Appropriate combinations or mixtures of such surfactants may also be used according to the present invention.

Many of these surfactants may also serve as emulsifiers in formulations of the present invention.

Other suitable emulsifiers for use in the formulations of the present invention include, but are not limited to, behentrimonium methosulfate-cetearyl alcohol, non-ionic emulsifiers like emulsifying wax, polyoxyethylene oleyl ether, PEG-40 stearate, cetostearyl alcohol (cetearyl alcohol), ceteareth-12, ceteareth-20, ceteareth-30, ceteareth alcohol, Ceteth-20 (Ceteth-20 is the polyethylene glycol ether of cetyl alcohol where n has an average value of 20), oleic acid, oleyl alcohol, glyceryl stearate, PEG-100 stearate, glyceryl stearate and PEG-100 stearate, ceramide 2, ceramide 3, stearic acid, cholesterol, steareth-2, and steareth-20, or combinations/mixtures thereof, as well as cationic emulsifiers like stearamidopropyl dimethylamine and behentrimonium methosulfate, or combinations/mixtures thereof.

### 4. Moisturizers, Emollients, and Humectants

One of the most important aspects of topical products in general, and cosmetic products in particular, is the consumer's perception of the aesthetic qualities of a product. For example, while white petrolatum is an excellent moisturizer and skin protectant, it is rarely used alone, especially on the face, because it is greasy, sticky, does not rub easily into the skin and may soil clothing. Consumers highly value products which are aesthetically elegant and have an acceptable tactile feel and performance on their skin.

Suitable moisturizers for use in the formulations of the present invention include, but are not limited to, lactic acid and other hydroxy acids and their salts, glycerol, propylene glycol, butylene glycol, sodium PCA, sodium hyaluronate, Carbowax 200, Carbowax 400, and Carbowax 800.

Suitable emollients or humectants for use in the formulations of the present invention include, but are not limited to, cetyl palmitate, glycerol (glycerin), PPG-15 stearyl ether, lanolin alcohol, lanolin, lanolin derivatives, cholesterol, petrolatum, isostearyl neopentanoate, octyl stearate, mineral oil, isocetyl stearate, myristyl myristate, octyl dodecanol, 2-ethylhexyl palmitate (octyl palmitate), dimethicone, phenyl trimethicone, cyclomethicone, C₁₂-C₁₅ alkyl benzoates, dimethiconol, propylene glycol, Theobroma grandiflorum seed butter, ceramides (e.g., ceramide 2 or ceramide 3), hydroxypropyl bispalmitamide MEA, hydroxypropyl bislauramide MEA, hydroxypropyl bisisostearamide MEA, 1,3-bis(N-2-(hydroxyethyl)stearoylamino)-2-hydroxy propane, bis-hydroxyethyl tocopherylsuccinoylamido hydroxypropane, urea, aloe, allantoin, glycyrrhetinic acid, safflower oil, oleyl alcohol, oleic acid, stearic acid, dicaprylate/dicaprate, diethyl sebacate, isostearyl alcohol, pentylene glycol, and 1,3-bis(N-2-(hydroxyethyl)palmitoylamino)-2-hydroxypropane.

In addition, appropriate combinations and mixtures of any of these moisturizing agents and emollients may be used in accordance with the present invention.

### 5. Preservatives and Antioxidants

The composition may further include components adapted to improve the stability or effectiveness of the applied formulation.

Suitable preservatives for use in the present invention include, but are not limited to: ureas, such as imidazolidinyl urea and diazolidinyl urea; phenoxyethanol; sodium methyl paraben, methylparaben, ethylparaben, and propylparaben; potassium sorbate; sodium benzoate; sorbic acid; benzoic acid; formaldehyde; citric acid; sodium citrate; chlorine dioxide; quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; piroctone olamine; Vitis vinifera seed oil; and alcoholic agents, for example, chlorobutanol, dichlorobenzyl alcohol, phenylethyl alcohol, and benzyl alcohol.

Suitable antioxidants include, but are not limited to, ascorbic acid and its esters, sodium bisulfite, butylated hydroxytoluene, butylated hydroxyanisole, tocopherols (such as α-tocopherol), tocopheryl acetate, sodium ascorbate/ascorbic acid, ascorbyl palmitate, propyl gallate, and chelating agents like EDTA (e.g., disodium EDTA), citric acid, and sodium citrate.

In certain embodiments, antioxidants or preservatives of the present invention may also function as a moisturizer or emollient, for example.

In addition, combinations or mixtures of these preservatives or anti-oxidants may also be used in the formulations of the present invention.

### 6. Active Agents

The active agent may be any material that has a desired effect when applied topically to a mammal, particularly a human. Suitable classes of active agents include, but are not limited to, antibiotic agents, antimicrobial agents, anti-acne agents, antibacterial agents, antifungal agents, antiviral agents, steroidal anti-inflammatory agents, non-steroidal anti-inflammatory agents, anesthetic agents, antipruriginous agents, antiprotozoal agents, anti-oxidants, antihistamines, vitamins, and hormones. Mixtures of any of these active agents may also be employed. Additionally, dermatologically-acceptable salts and esters of any of these agents may be employed.

6.1 Antibiotics

Representative antibiotics include, without limitation, benzoyl peroxide, alfa terpineol, octopirox, erythromycin, zinc, tetracyclin, triclosan, azelaic acid and its derivatives, phenoxy ethanol and phenoxy propanol, ethyl acetate, clindamycin (e.g., clindamycin phosphate) and meclocycline; sebostats such as flavinoids; alpha and beta hydroxy acids; and bile salts such as scymnol sulfate and its derivatives, deoxycholate and cholate. The antibiotic can be an antifungal agent. Suitable antifungal agents include, but are not limited to, clotrimazole, econazole, ketoconazole, itraconazole, miconazole, oxiconazole, sulconazole, butenafine, naftifine, terbinafine, undecylinic acid, tolnaftate, and nystatin. Mixtures of these antibiotic agents may also be employed. Additionally, dermatologically-acceptable salts and esters of any of these agents may be employed.

6.2 Non-Steroidal Anti-Inflammatory Agents

Representative examples of non-steroidal anti-inflammatory agents include, without limitation, oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac, fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed, as well as the dermatologically acceptable salts and esters of these agents. For example, etofenamiate, a flufenamic acid derivative, is particularly useful for topical application.

6.3 Steroidal Anti-Inflammatory Agents

Representative examples of steroidal anti-inflammatory drugs include, without limitation, corticosteroids such as hydrocortisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters (including betamethasone dipropionate), chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

6.4 Anesthetics

Suitable anesthetics include the aminoacylanilide compounds such as lidocaine, prilocalne, bupivacaine, levo-bupivacaine, ropivacaine, mepivacaine and related local anesthetic compounds having various substituents on the ring system or amine nitrogen; the aminoalkyl benzoate compounds, such as procaine, chloroprocaine, propoxycaine, hexylcaine, tetracaine, cyclomethycaine, benoxinate, butacaine, proparacaine, butamben, and related local anesthetic compounds; cocaine and related local anesthetic compounds; amino carbonate compounds such as diperodon and related local anesthetic compounds; N-phenylamidine compounds such as phenacaine and related anesthetic compounds; N-aminoalkyl amide compounds such as dibucaine and related local anesthetic compounds; aminoketone compounds such as falicaine, dyclonine and related local anesthetic compounds; and amino ether compounds such as pramoxine, dimethisoquien, and related local anesthetic compounds; and para-amino benzoic acid esters such as benzocaine. Other suitable local anesthetics include ketocaine, dibucaine, amethocaine, propanacaine, and propipocaine.

6.5 Antimicrobial Agents

Suitable antimicrobial agents include, but are not limited to, antibacterial, antifungal, antiprotozoal and antiviral agents, such as beta-lactam drugs, quinolone drugs, ciprofloxacin, norfloxacin, tetracycline, erythromycin, amikacin, triclosan, doxycycline, capreomycin, chlorhexidine, chlortetracycline, oxytetracycline, clindamycin (e.g., clindamycin phosphate), ethambutol, metronidazole, pentamidine, gentamicin, kanamycin, lineomycin, methacycline, methenamine, minocycline, neomycin, netilmicin, streptomycin, tobramycin, and miconazole. Also included are tetracycline hydrochloride, famesol, erythromycin estolate, erythromycin stearate (salt), amikacin sulfate, doxycycline hydrochloride, chlorhexidine gluconate, chlorhexidine hydrochloride, chlortetracycline hydrochloride, oxytetracycline hydrochloride, clindamycin hydrochloride, clindamycin phosphate, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, triclosan, octopirox, nystatin, tolnaftate, clotrimazole, anidulafungin, micafungin, voriconazole, lanoconazole, ciclopirox and mixtures thereof.

6.6 Keratolytic Agents

Suitable keratolytic agents include, but are not limited to, urea, salicylic acid, papain, sulfur, glycolic acid, pyruvic acid, resorcinol, N-acetylcysteine, retinoids such as retinoic acid (e.g., tretinoin) and its derivatives (e.g., cis and trans, esters), alpha hydroxy acids, beta hydroxy acids, coal tar, and combinations thereof.

### 7. Purging Gases

In one embodiment, the air in the container charged with the composition is replaced by an inert gas. In certain embodiments, the inert gas is selected from the group consisting of argon, nitrogen, and mixtures thereof.

### 8. Buffer Salts

Suitable buffer salts are well-known in the art. Examples of suitable buffer salts include, but are not limited to sodium citrate, citric acid, sodium phosphate monobasic, sodium phosphate dibasic, sodium phosphate tribasic, potassium phosphate monobasic, potassium phosphate dibasic, and potassium phosphate tribasic.

### 9. Viscosity Modifiers

Suitable viscosity adjusting agents (i.e., thickening and thinning agents) for use in the formulations of the present invention include, but are not limited to, protective colloids or non-ionic gums such as hydroxyethylcellulose, xanthan gum, and sclerotium gum, as well as magnesium aluminum silicate, silica, microcrystalline wax, beeswax, paraffin, and cetyl palmitate. In addition, appropriate combinations or mixtures of these viscosity adjusters may be utilized according to the present invention.

### 10. Additional Constituents

Additional constituents suitable for incorporation into the emulsions of the present invention include, but are not limited to: skin protectants, adsorbents, demulcents, emollients, moisturizers, sustained release materials, solubilizing agents, skin-penetration agents, skin soothing agents, deodorant agents, antiperspirants, sun screening agents, sunless tanning agents, vitamins, hair conditioning agents, anti-irritants, anti-aging agents, abrasives, absorbents, anti-caking agents, anti-static agents, astringents (e.g., witch hazel, alcohol, and herbal extracts such as chamomile extract), binders/excipients, buffering agents, chelating agents, film forming agents, conditioning agents, opacifying agents, lipids, immunomodulators, and pH adjusters (e.g., citric acid, sodium hydroxide, and sodium phosphate).

For example, lipids normally found in healthy skin (or their functional equivalents) may be incorporated into the emulsions of the present invention. In certain embodiments, the lipid is selected from the group consisting of ceramides, cholesterol, and free fatty acids. Examples of lipids include, but are not limited to, ceramide 1, ceramide 2, ceramide 3, ceramide 4, ceramide 5, ceramide 6, hydroxypropyl bispalmitamide MEA, and hydroxypropyl bislauramide MEA, and combinations thereof.

Examples of skin soothing agents include, but are not limited to, allantoin, aloe, avocado oil, green tea extract, hops extract, chamomile extract, colloidal oatmeal, calamine, cucumber extract, and combinations thereof.

Examples of vitamins include, but are not limited to, vitamins A, D, E, K, and combinations thereof. Vitamin analogues are also contemplated; for example the vitamin D analogues calcipotriene or calcipotriol.

Examples of sunscreens include, but are not limited to, p-aminobenzoic acid, avobenzone, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, titanium dioxide, trolamine salicylate, zinc oxide, 4-methylbenzylidene camphor, methylene bis-benzotriazolyl tetramethylbutylphenol, bis-ethylhexyloxyphenol methoxyphenyl triazine, terephthalylidene dicamphor sulfonic acid, drometrizole trisiloxane, disodium phenyl dibenzimidazole tetrasulfonate, diethylamino hydroxybenzoyl hexyl benzoate, octyl triazone, diethylhexyl butamido triazone, polysilicone-15, and combinations thereof.

Suitable fragrances and colors may be used in the formulations of the present invention. Examples of fragrances and colors suitable for use in topical products are known in the art.

Suitable immunomodulators include, but are not limited to, tetrachlorodecaoxide, deoxycholic acid, tacrolimus, pimecrolimus, and beta-glucan.

Often, one constituent of a composition may accomplish several functions. In one embodiment, the present invention relates to constituents that may act as a lubricant, an emollient, or a skin-penetrating agent. In one embodiment, the multi-functional constituent is socetyl stearate, isopropyl isostearate, isopropyl palmitate, or isopropyl myristate.

### Exemplary Emulsions of the Invention

In certain embodiments, the invention relates to an emulsion, comprising:

water,

an emulsifier or surfactant,

a moisturizer or emollient,

an antioxidant or preservative, and

an active agent.

In certain embodiments, the invention relates to an emulsion, consisting essentially of:

water,

an emulsifier or surfactant,

a moisturizer or emollient,

an antioxidant or preservative, and

an active agent.

In certain embodiments, the invention relates to an emulsion, consisting of:

water,

an emulsifier or surfactant,

a moisturizer or emollient,

an antioxidant or preservative, and

an active agent.

In certain embodiments, the invention relates to an emulsion, comprising:

water,

an emulsifier or surfactant,

a moisturizer or emollient,

an antioxidant or preservative,

a pH adjuster, and

an active agent.

In certain embodiments, the invention relates to an emulsion, consisting essentially of:

water,

an emulsifier or surfactant,

a moisturizer or emollient,

an antioxidant or preservative,

a pH adjuster, and

an active agent.

In certain embodiments, the invention relates to an emulsion, consisting of:

water,

an emulsifier or surfactant,

a moisturizer or emollient,

an antioxidant or preservative,

a pH adjuster, and

an active agent.

In certain embodiments, the invention relates to an emulsion, comprising:

water,

an emulsifier or surfactant,

a moisturizer or emollient,

an antioxidant or preservative,

a pH adjuster,

a buffer, and

an active agent.

In certain embodiments, the invention relates to an emulsion, consisting essentially of:

water,

an emulsifier or surfactant,

a moisturizer or emollient,

an antioxidant or preservative,

a pH adjuster,

a buffer, and

an active agent.

In certain embodiments, the invention relates to an emulsion, consisting of:

water,

an emulsifier or surfactant,

a moisturizer or emollient,

an antioxidant or preservative,

a pH adjuster,

a buffer, and

an active agent.

In certain embodiments, the invention relates to an emulsion, comprising:
water, from about 24% to about 90% by weight of the emulsion;
sodium hyaluronate, from about 0.05% to about 1.50% by weight of the emulsion;
glycerin, from about 2.5% to about 10% by weight of the emulsion;
ethylhexyl palmitate, from about 3% to about 9% by weight of the emulsion;
cetostearyl alcohol, from about 0.5% to about 5% by weight of the emulsion;
dicetyl phosphate, from about 1% to about 3% by weight of the emulsion;
ceteth-10 phosphate, from about 0.5% to about 1.5% by weight of the emulsion;
propylene glycol, from about 1% to about 4% by weight of the emulsion;
theobroma grandiflorum seed butter, from about 1% to about 3% by weight of the emulsion;
dimethicone, from about 0.2% to about 2% by weight of the emulsion;
petrolatum, from about 0.5% to about 6% by weight of the emulsion;
polyethylene glycol octadecyl ether, from about 0.2% to about 3% by weight of the emulsion;
tocopheryl acetate, from about 0.2% to about 0.8% by weight of the emulsion;
1,3-bis(N-2-(hydroxyethyl)palmitoylamino)-2-hydroxypropane, from about 0.2% to about 0.8% by weight of the emulsion;
methylparaben, from about 0.01% to about 0.5% by weight of the emulsion;
sodium hydroxide, from about 0.01% to about 0.3% by weight of the emulsion;
propylparaben, from about 0.05% to about 0.2% by weight of the emulsion; and
disodium EDTA, from about 0.05% to about 0.2% by weight of the emulsion.

In certain embodiments, the invention relates to an emulsion, consisting essentially of:
water, from about 24% to about 90% by weight of the emulsion;
sodium hyaluronate, from about 0.05% to about 1.5% by weight of the emulsion;
glycerin, from about 2.5% to about 10% by weight of the emulsion;
ethylhexyl palmitate, from about 3% to about 9% by weight of the emulsion;
cetostearyl alcohol, from about 0.5% to about 5% by weight of the emulsion;
dicetyl phosphate, from about 1% to about 3% by weight of the emulsion;
ceteth-10 phosphate, from about 0.5% to about 1.5% by weight of the emulsion;
propylene glycol, from about 1% to about 4% by weight of the emulsion;
theobroma grandiflorum seed butter, from about 1% to about 3% by weight of the emulsion;
dimethicone, from about 0.2% to about 2% by weight of the emulsion;
petrolatum, from about 0.5% to about 6% by weight of the emulsion;
polyethylene glycol octadecyl ether, from about 0.2% to about 3% by weight of the emulsion;
tocopheryl acetate, from about 0.2% to about 0.8% by weight of the emulsion;
1,3-bis(N-2-(hydroxyethyl)palmitoylamino)-2-hydroxypropane, from about 0.2% to about 0.8% by weight of the emulsion;
methylparaben, from about 0.01% to about 0.5% by weight of the emulsion;
sodium hydroxide, from about 0.01% to about 0.3% by weight of the emulsion;
propylparaben, from about 0.05% to about 0.2% by weight of the emulsion; and
disodium EDTA, from about 0.05% to about 0.2% by weight of the emulsion.

In certain embodiments, the invention relates to an emulsion, consisting of:
water, from about 24% to about 90% by weight of the emulsion;
sodium hyaluronate, from about 0.05% to about 1.5% by weight of the emulsion;
glycerin, from about 2.5% to about 10% by weight of the emulsion;
ethylhexyl palmitate, from about 3% to about 9% by weight of the emulsion;
cetostearyl alcohol, from about 0.5% to about 5% by weight of the emulsion;
dicetyl phosphate, from about 1% to about 3% by weight of the emulsion;
ceteth-10 phosphate, from about 0.5% to about 1.5% by weight of the emulsion;
propylene glycol, from about 1% to about 4% by weight of the emulsion;
theobroma grandiflorum seed butter, from about 1% to about 3% by weight of the emulsion;
dimethicone, from about 0.2% to about 2% by weight of the emulsion;
petrolatum, from about 0.5% to about 6% by weight of the emulsion;
Polyethylene glycol octadecyl ether, from about 0.2% to about 3% by weight of the emulsion;
tocopheryl acetate, from about 0.2% to about 0.8% by weight of the emulsion;
1,3-bis(N-2-(hydroxyethyl)palmitoylamino)-2-hydroxypropane, from about 0.2% to about 0.8% by weight of the emulsion;
methylparaben, from about 0.01% to about 0.5% by weight of the emulsion;
sodium hydroxide, from about 0.01% to about 0.3% by weight of the emulsion;
propylparaben, from about 0.05% to about 0.2% by weight of the emulsion; and
disodium EDTA, from about 0.05% to about 0.2% by weight of the emulsion.

In certain embodiments, the invention relates to an emulsion, comprising:
water, from about 24.00% to about 90.00% by weight of the emulsion;
sodium hyaluronate, from about 0.05% to about 1.50% by weight of the emulsion;
glycerin, from about 2.50% to about 10.00% by weight of the emulsion;
ethylhexyl palmitate, from about 3.00% to about 9.00% by weight of the emulsion;
cetostearyl alcohol, from about 0.50% to about 5.00% by weight of the emulsion;
dicetyl phosphate, from about 1.00% to about 3.00% by weight of the emulsion;
ceteth-10 phosphate, from about 0.50% to about 1.50% by weight of the emulsion;
propylene glycol, from about 1.00% to about 4.00% by weight of the emulsion;
theobroma grandiflorum seed butter, from about 1.00% to about 3.00% by weight of the emulsion;
dimethicone, from about 0.20% to about 2.00% by weight of the emulsion;
petrolatum, from about 0.50% to about 6.00% by weight of the emulsion;
Polyethylene glycol octadecyl ether, from about 0.20% to about 3.00% by weight of the emulsion;
tocopheryl acetate, from about 0.20% to about 0.80% by weight of the emulsion;
1,3-bis(N-2-(hydroxyethyl)palmitoylamino)-2-hydroxypropane, from about 0.20% to about 0.80% by weight of the emulsion;
methylparaben, from about 0.01% to about 0.50% by weight of the emulsion;
sodium hydroxide, from about 0.01% to about 0.30% by weight of the emulsion;
propylparaben, from about 0.05% to about 0.20% by weight of the emulsion; and
disodium EDTA, from about 0.05% to about 0.20% by weight of the emulsion.

In certain embodiments, the invention relates to an emulsion, consisting essentially of:
water, from about 24.00% to about 90.00% by weight of the emulsion;
sodium hyaluronate, from about 0.05% to about 1.50% by weight of the emulsion;
glycerin, from about 2.50% to about 10.00% by weight of the emulsion;
ethylhexyl palmitate, from about 3.00% to about 9.00% by weight of the emulsion;
cetostearyl alcohol, from about 0.50% to about 5.00% by weight of the emulsion;
dicetyl phosphate, from about 1.00% to about 3.00% by weight of the emulsion;
ceteth-10 phosphate, from about 0.50% to about 1.50% by weight of the emulsion;
propylene glycol, from about 1.00% to about 4.00% by weight of the emulsion;
theobroma grandiflorum seed butter, from about 1.00% to about 3.00% by weight of the emulsion;
dimethicone, from about 0.20% to about 2.00% by weight of the emulsion;
petrolatum, from about 0.50% to about 6.00% by weight of the emulsion;
Polyethylene glycol octadecyl ether, from about 0.20% to about 3.00% by weight of the emulsion;
tocopheryl acetate, from about 0.20% to about 0.80% by weight of the emulsion;
1,3-bis(N-2-(hydroxyethyl)palmitoylamino)-2-hydroxypropane, from about 0.20% to about 0.80% by weight of the emulsion;
methylparaben, from about 0.01% to about 0.50% by weight of the emulsion;
sodium hydroxide, from about 0.01% to about 0.30% by weight of the emulsion;
propylparaben, from about 0.05% to about 0.20% by weight of the emulsion; and
disodium EDTA, from about 0.05% to about 0.20% by weight of the emulsion.

In certain embodiments, the invention relates to an emulsion, consisting of:
water, from about 24.00% to about 90.00% by weight of the emulsion;
sodium hyaluronate, from about 0.05% to about 1.50% by weight of the emulsion;
glycerin, from about 2.50% to about 10.00% by weight of the emulsion;
ethylhexyl palmitate, from about 3.00% to about 9.00% by weight of the emulsion;
cetostearyl alcohol, from about 0.50% to about 5.00% by weight of the emulsion;
dicetyl phosphate, from about 1.00% to about 3.00% by weight of the emulsion;
ceteth-10 phosphate, from about 0.50% to about 1.50% by weight of the emulsion;
propylene glycol, from about 1.00% to about 4.00% by weight of the emulsion;
theobroma grandiflorum seed butter, from about 1.00% to about 3.00% by weight of the emulsion;
dimethicone, from about 0.20% to about 2.00% by weight of the emulsion;
petrolatum, from about 0.50% to about 6.00% by weight of the emulsion;
Polyethylene glycol octadecyl ether, from about 0.20% to about 3.00% by weight of the emulsion;
tocopheryl acetate, from about 0.20% to about 0.80% by weight of the emulsion;
1,3-bis(N-2-(hydroxyethyl)palmitoylamino)-2-hydroxypropane, from about 0.20% to about 0.80% by weight of the emulsion;
methylparaben, from about 0.01% to about 0.50% by weight of the emulsion;
sodium hydroxide, from about 0.01% to about 0.30% by weight of the emulsion;
propylparaben, from about 0.05% to about 0.20% by weight of the emulsion; and
disodium EDTA, from about 0.05% to about 0.20% by weight of the emulsion.

In certain embodiments, the invention relates to an emulsion, comprising:
water, from about 24% to about 90% by weight of the emulsion;
benzoyl peroxide, from about 0.05% to about 15% by weight of the emulsion;
glycerin, from about 2.5% to about 10% by weight of the emulsion;
propylene glycol, from about 1% to about 4% by weight of the emulsion;
cetostearyl alcohol, from about 0.5% to about 5% by weight of the emulsion;
emulsifying wax, from about 0.5% to about 5% by weight of the emulsion;
dimethicone, from about 0.2% to about 2% by weight of the emulsion;
sodium citrate, from about 0.3% to about 0.9% by weight of the emulsion;
Polyethylene glycol octadecyl ether, from about 0.2% to about 3% by weight of the emulsion;
methylparaben, from about 0.01% to about 0.5% by weight of the emulsion;
C₁₂-C₁₅ alkyl benzoates, from about 0.1% to about 0.3% by weight of the emulsion;
propylparaben, from about 0.05% to about 0.2% by weight of the emulsion;
disodium EDTA, from about 0.05% to about 0.2% by weight of the emulsion;
butylated hydroxytoluene, from about 0.01% to about 0.08% by weight of the emulsion; and
citric acid, from about 0.02% to about 0.08% by weight of the emulsion.

In certain embodiments, the invention relates to an emulsion, consisting essentially of:
water, from about 24% to about 90% by weight of the emulsion;
benzoyl peroxide, from about 0.05% to about 15% by weight of the emulsion;
glycerin, from about 2.5% to about 10% by weight of the emulsion;
propylene glycol, from about 1% to about 4% by weight of the emulsion;
cetostearyl alcohol, from about 0.5% to about 5% by weight of the emulsion;
emulsifying wax, from about 0.5% to about 5% by weight of the emulsion;
dimethicone, from about 0.2% to about 2% by weight of the emulsion;
sodium citrate, from about 0.3% to about 0.9% by weight of the emulsion;
Polyethylene glycol octadecyl ether, from about 0.2% to about 3% by weight of the emulsion;
methylparaben, from about 0.01% to about 0.5% by weight of the emulsion;
C₁₂-C₁₅ alkyl benzoates, from about 0.1% to about 0.3% by weight of the emulsion;
propylparaben, from about 0.05% to about 0.2% by weight of the emulsion;
disodium EDTA, from about 0.05% to about 0.2% by weight of the emulsion;
butylated hydroxytoluene, from about 0.01% to about 0.08% by weight of the emulsion; and
citric acid, from about 0.02% to about 0.08% by weight of the emulsion.

In certain embodiments, the invention relates to an emulsion, consisting of:
water, from about 24% to about 90% by weight of the emulsion;
benzoyl peroxide, from about 0.05% to about 15% by weight of the emulsion;
glycerin, from about 2.5% to about 10% by weight of the emulsion;
propylene glycol, from about 1% to about 4% by weight of the emulsion;
cetostearyl alcohol, from about 0.5% to about 5% by weight of the emulsion;
emulsifying wax, from about 0.5% to about 5% by weight of the emulsion;
dimethicone, from about 0.2% to about 2% by weight of the emulsion;
sodium citrate, from about 0.3% to about 0.9% by weight of the emulsion;
Polyethylene glycol octadecyl ether, from about 0.2% to about 3% by weight of the emulsion;
methylparaben, from about 0.01% to about 0.5% by weight of the emulsion;
C₁₂-C₁₅ alkyl benzoates, from about 0.1% to about 0.3% by weight of the emulsion;
propylparaben, from about 0.05% to about 0.2% by weight of the emulsion;
disodium EDTA, from about 0.05% to about 0.2% by weight of the emulsion;
butylated hydroxytoluene, from about 0.01% to about 0.08% by weight of the emulsion; and
citric acid, from about 0.02% to about 0.08% by weight of the emulsion.

In certain embodiments, the invention relates to an emulsion, comprising:
water, from about 24.00% to about 90.00% by weight of the emulsion;
benzoyl peroxide, from about 0.05% to about 15.00% by weight of the emulsion;
glycerin, from about 2.50% to about 10.00% by weight of the emulsion;
propylene glycol, from about 1.00% to about 4.00% by weight of the emulsion;
cetostearyl alcohol, from about 0.50% to about 5.00% by weight of the emulsion;
emulsifying wax, from about 0.50% to about 5.00% by weight of the emulsion;
dimethicone, from about 0.20% to about 2.00% by weight of the emulsion;
sodium citrate, from about 0.30% to about 0.90% by weight of the emulsion;
Polyethylene glycol octadecyl ether, from about 0.20% to about 3.00% by weight of the emulsion;
methylparaben, from about 0.01% to about 0.50% by weight of the emulsion;
C₁₂-C₁₅ alkyl benzoates, from about 0.10% to about 0.30% by weight of the emulsion;
propylparaben, from about 0.05% to about 0.20% by weight of the emulsion;
disodium EDTA, from about 0.05% to about 0.20% by weight of the emulsion;
butylated hydroxytoluene, from about 0.01% to about 0.08% by weight of the emulsion; and
citric acid, from about 0.02% to about 0.08% by weight of the emulsion.

In certain embodiments, the invention relates to an emulsion, consisting essentially of:
water, from about 24.00% to about 90.00% by weight of the emulsion;
benzoyl peroxide, from 0.05% to about 15.00% by weight of the emulsion;
glycerin, from about 2.50% to about 10.00% by weight of the emulsion;
propylene glycol, from about 1.00% to about 4.00% by weight of the emulsion;
cetostearyl alcohol, from about 0.50% to about 5.00% by weight of the emulsion;
emulsifying wax, from about 0.50% to about 5.00% by weight of the emulsion;
dimethicone, from about 0.20% to about 2.00% by weight of the emulsion;
sodium citrate, from about 0.30% to about 0.90% by weight of the emulsion;
Polyethylene glycol octadecyl ether, from about 0.20% to about 3.00% by weight of the emulsion;
methylparaben, from about 0.01% to about 0.50% by weight of the emulsion;
C₁₂-C₁₅ alkyl benzoates, from about 0.10% to about 0.30% by weight of the emulsion;
propylparaben, from about 0.05% to about 0.20% by weight of the emulsion;
disodium EDTA, from about 0.05% to about 0.20% by weight of the emulsion;
butylated hydroxytoluene, from about 0.01% to about 0.08% by weight of the emulsion; and
citric acid, from about 0.02% to about 0.08% by weight of the emulsion.

In certain embodiments, the invention relates to an emulsion, consisting of:
water, from about 24.00% to about 90.00% by weight of the emulsion;
benzoyl peroxide, from about 0.05% to about 15.00% by weight of the emulsion;
glycerin, from about 2.50% to about 10.00% by weight of the emulsion;
propylene glycol, from about 1.00% to about 4.00% by weight of the emulsion;
cetostearyl alcohol, from about 0.50% to about 5.00% by weight of the emulsion;
emulsifying wax, from about 0.50% to about 5.00% by weight of the emulsion;
dimethicone, from about 0.20% to about 2.00% by weight of the emulsion;
sodium citrate, from about 0.30% to about 0.90% by weight of the emulsion;
Polyethylene glycol octadecyl ether, from about 0.20% to about 3.00% by weight of the emulsion;
methylparaben, from about 0.01% to about 0.50% by weight of the emulsion;
C₁₂-C₁₅ alkyl benzoates, from about 0.10% to about 0.30% by weight of the emulsion;
propylparaben, from about 0.05% to about 0.20% by weight of the emulsion;
disodium EDTA, from about 0.05% to about 0.20% by weight of the emulsion;
butylated hydroxytoluene, from about 0.01% to about 0.08% by weight of the emulsion; and
citric acid, from about 0.02% to about 0.08% by weight of the emulsion.

In certain embodiments, the invention relates to an emulsion, comprising:
water, from about 24% to about 90% by weight of the emulsion;
oleic acid, from about 1.5% to about 14% by weight of the emulsion;
glycerin, from about 2.5% to about 10% by weight of the emulsion;
alfa terpineol, from about 1.5% to about 4.5% by weight of the emulsion;
pentylene glycol, from about 1% to about 4% by weight of the emulsion;
cetostearyl alcohol, from about 0.5% to about 5% by weight of the emulsion;
emulsifying wax, from about 0.5% to about 5% by weight of the emulsion;
sodium phosphate monobasic, from about 0.5% to about 1.5% by weight of the emulsion;
Polyethylene glycol octadecyl ether, from about 0.2% to about 3% by weight of the emulsion;
Poloxamer 188, from about 0.01% to about 2% by weight of the emulsion;
methylparaben, from about 0.01% to about 0.5% by weight of the emulsion;
sodium hydroxide, from about 0.01% to about 0.3% by weight of the emulsion;
propylparaben, from about 0.05% to about 0.2% by weight of the emulsion;
disodium EDTA, from about 0.05% to about 0.2% by weight of the emulsion;
butylated hydroxytoluene, from about 0.01% to about 0.08% by weight of the emulsion; and
tretinoin, from about 0.05% to about 1% by weight of the emulsion.

In certain embodiments, the invention relates to an emulsion, consisting essentially of:
water, from about 24% to about 90% by weight of the emulsion;
oleic acid, from about 1.5% to about 14% by weight of the emulsion;
glycerin, from about 2.5% to about 10% by weight of the emulsion;
alfa terpineol, from about 1.5% to about 4.5% by weight of the emulsion;
pentylene glycol, from about 1% to about 4% by weight of the emulsion;
cetostearyl alcohol, from about 0.5% to about 5% by weight of the emulsion;
emulsifying wax, from about 0.5% to about 5% by weight of the emulsion;
sodium phosphate monobasic, from about 0.5% to about 1.5% by weight of the emulsion;
Polyethylene glycol octadecyl ether, from about 0.2% to about 3% by weight of the emulsion;
Poloxamer 188, from about 0.01% to about 2% by weight of the emulsion;
methylparaben, from about 0.01% to about 0.5% by weight of the emulsion;
sodium hydroxide, from about 0.01% to about 0.3% by weight of the emulsion;
propylparaben, from about 0.05% to about 0.2% by weight of the emulsion;
disodium EDTA, from about 0.05% to about 0.2% by weight of the emulsion;
butylated hydroxytoluene, from about 0.01% to about 0.08% by weight of the emulsion; and
tretinoin, from about 0.05% to about 1% by weight of the emulsion.

In certain embodiments, the invention relates to an emulsion, consisting of:
water, from about 24% to about 90% by weight of the emulsion;
oleic acid, from about 1.5% to about 14% by weight of the emulsion;
glycerin, from about 2.5% to about 10% by weight of the emulsion;
alfa terpineol, from about 1.5% to about 4.5% by weight of the emulsion;
pentylene glycol, from about 1% to about 4% by weight of the emulsion;
cetostearyl alcohol, from about 0.5% to about 5% by weight of the emulsion;
emulsifying wax, from about 0.5% to about 5% by weight of the emulsion;
sodium phosphate monobasic, from about 0.5% to about 1.5% by weight of the emulsion;
Polyethylene glycol octadecyl ether, from about 0.2% to about 3% by weight of the emulsion;
Poloxamer 188, from about 0.01% to about 2% by weight of the emulsion;
methylparaben, from about 0.01% to about 0.5% by weight of the emulsion;
sodium hydroxide, from about 0.01% to about 0.3% by weight of the emulsion;
propylparaben, from about 0.05% to about 0.2% by weight of the emulsion;
disodium EDTA, from about 0.05% to about 0.2% by weight of the emulsion;
butylated hydroxytoluene, from about 0.01% to about 0.08% by weight of the emulsion; and
tretinoin, from about 0.05% to about 1% by weight of the emulsion.

In certain embodiments, the invention relates to an emulsion, comprising:
water, from about 24.00% to about 90.00% by weight of the emulsion;
oleic acid, from about 1.50% to about 14.00% by weight of the emulsion;
glycerin, from about 2.50% to about 10.00% by weight of the emulsion;
alfa terpineol, from about 1.50% to about 4.50% by weight of the emulsion;
pentylene glycol, from about 1.00% to about 4.00% by weight of the emulsion;
cetostearyl alcohol, from about 0.50% to about 5.00% by weight of the emulsion;
emulsifying wax, from about 0.50% to about 5.00% by weight of the emulsion;
sodium phosphate monobasic, from about 0.50% to about 1.50% by weight of the emulsion;
Polyethylene glycol octadecyl ether, from about 0.20% to about 3.00% by weight of the emulsion;
Poloxamer 188, from about 0.01% to about 2.00% by weight of the emulsion;
methylparaben, from about 0.01% to about 0.50% by weight of the emulsion;
sodium hydroxide, from about 0.01% to about 0.30% by weight of the emulsion;
propylparaben, from about 0.05% to about 0.20% by weight of the emulsion;
disodium EDTA, from about 0.05% to about 0.20% by weight of the emulsion;
butylated hydroxytoluene, from about 0.01% to about 0.08% by weight of the emulsion; and
tretinoin, from about 0.05% to about 1.00% by weight of the emulsion.

In certain embodiments, the invention relates to an emulsion, consisting essentially of:
water, from about 24.00% to about 90.00% by weight of the emulsion;
oleic acid, from about 1.50% to about 14.00% by weight of the emulsion;
glycerin, from about 2.50% to about 10.00% by weight of the emulsion;
alfa terpineol, from about 1.50% to about 4.50% by weight of the emulsion;
pentylene glycol, from about 1.00% to about 4.00% by weight of the emulsion;
cetostearyl alcohol, from about 0.50% to about 5.00% by weight of the emulsion;
emulsifying wax, from about 0.50% to about 5.00% by weight of the emulsion;
sodium phosphate monobasic, from about 0.50% to about 1.50% by weight of the emulsion;
Polyethylene glycol octadecyl ether, from about 0.20% to about 3.00% by weight of the emulsion;
Poloxamer 188, from about 0.01% to about 2.00% by weight of the emulsion;
methylparaben, from about 0.01% to about 0.50% by weight of the emulsion;
sodium hydroxide, from about 0.01% to about 0.30% by weight of the emulsion;
propylparaben, from about 0.05% to about 0.20% by weight of the emulsion;
disodium EDTA, from about 0.05% to about 0.20% by weight of the emulsion;
butylated hydroxytoluene, from about 0.01% to about 0.08% by weight of the emulsion; and
tretinoin, from about 0.05% to about 1.00% by weight of the emulsion.

In certain embodiments, the invention relates to an emulsion, consisting of:
water, from about 24.00% to about 90.00% by weight of the emulsion;
oleic acid, from about 1.50% to about 14.00% by weight of the emulsion;
glycerin, from about 2.50% to about 10.00% by weight of the emulsion;
alfa terpineol, from about 1.50% to about 4.50% by weight of the emulsion;
pentylene glycol, from about 1.00% to about 4.00% by weight of the emulsion;
cetostearyl alcohol, from about 0.50% to about 5.00% by weight of the emulsion;
emulsifying wax, from about 0.50% to about 5.00% by weight of the emulsion;
sodium phosphate monobasic, from about 0.50% to about 1.50% by weight of the emulsion;
Polyethylene glycol octadecyl ether, from about 0.20% to about 3.00% by weight of the emulsion;
Poloxamer 188, from about 0.01% to about 2.00% by weight of the emulsion;
methylparaben, from about 0.01% to about 0.50% by weight of the emulsion;
sodium hydroxide, from about 0.01% to about 0.30% by weight of the emulsion;
propylparaben, from about 0.05% to about 0.20% by weight of the emulsion;
disodium EDTA, from about 0.05% to about 0.20% by weight of the emulsion;
butylated hydroxytoluene, from about 0.01% to about 0.08% by weight of the emulsion; and
tretinoin, from about 0.05% to about 1.00% by weight of the emulsion.

In certain embodiments, the invention relates to an emulsion, comprising:
water, from about 24% to about 90% by weight of the emulsion;
oleic acid, from about 1.5% to about 14% by weight of the emulsion;
diethyl sebacate, from about 2% to about 12% by weight of the emulsion;
glycerin, from about 2.5% to about 10% by weight of the emulsion;
pentylene glycol, from about 1% to about 4% by weight of the emulsion;
cetostearyl alcohol, from about 0.5% to about 5% by weight of the emulsion;
emulsifying wax, from about 0.5% to about 5% by weight of the emulsion;
sodium phosphate monobasic, from about 0.5% to about 1.5% by weight of the emulsion;
Polyethylene glycol octadecyl ether, from about 0.2% to about 3% by weight of the emulsion;
Poloxamer 188, from about 0.01% to about 2% by weight of the emulsion;
methylparaben, from about 0.01% to about 0.5% by weight of the emulsion;
sodium hydroxide, from about 0.01% to about 0.3% by weight of the emulsion;
propylparaben, from about 0.05% to about 0.2% by weight of the emulsion;
disodium EDTA, from about 0.05% to about 0.2% by weight of the emulsion;
butylated hydroxytoluene, from about 0.01% to about 0.08% by weight of the emulsion; and
tretinoin, from about 0.05% to about 1% by weight of the emulsion.

In certain embodiments, the invention relates to an emulsion, consisting essentially of:
water, from about 24% to about 90% by weight of the emulsion;
oleic acid, from about 1.5% to about 14% by weight of the emulsion;
diethyl sebacate, from about 2% to about 12% by weight of the emulsion;
glycerin, from about 2.5% to about 10% by weight of the emulsion;
pentylene glycol, from about 1% to about 4% by weight of the emulsion;
cetostearyl alcohol, from about 0.5% to about 5% by weight of the emulsion;
emulsifying wax, from about 0.5% to about 5% by weight of the emulsion;
sodium phosphate monobasic, from about 0.5% to about 1.5% by weight of the emulsion;
Polyethylene glycol octadecyl ether, from about 0.2% to about 3% by weight of the emulsion;
Poloxamer 188, from about 0.01% to about 2% by weight of the emulsion;
methylparaben, from about 0.01% to about 0.5% by weight of the emulsion;
sodium hydroxide, from about 0.01% to about 0.3% by weight of the emulsion;
propylparaben, from about 0.05% to about 0.2% by weight of the emulsion;
disodium EDTA, from about 0.05% to about 0.2% by weight of the emulsion;
butylated hydroxytoluene, from about 0.01% to about 0.08% by weight of the emulsion; and
tretinoin, from about 0.05% to about 1% by weight of the emulsion.

In certain embodiments, the invention relates to an emulsion, consisting of:
water, from about 24% to about 90% by weight of the emulsion;
oleic acid, from about 1.5% to about 14% by weight of the emulsion;
diethyl sebacate, from about 2% to about 12% by weight of the emulsion;
glycerin, from about 2.5% to about 10% by weight of the emulsion;
pentylene glycol, from about 1% to about 4% by weight of the emulsion;
cetostearyl alcohol, from about 0.5% to about 5% by weight of the emulsion;
emulsifying wax, from about 0.5% to about 5% by weight of the emulsion;
sodium phosphate monobasic, from about 0.5% to about 1.5% by weight of the emulsion;
Polyethylene glycol octadecyl ether, from about 0.2% to about 3% by weight of the emulsion;
Poloxamer 188, from about 0.01% to about 2% by weight of the emulsion;
methylparaben, from about 0.01% to about 0.5% by weight of the emulsion;
sodium hydroxide, from about 0.01% to about 0.3% by weight of the emulsion;
propylparaben, from about 0.05% to about 0.2% by weight of the emulsion;
disodium EDTA, from about 0.05% to about 0.2% by weight of the emulsion;
butylated hydroxytoluene, from about 0.01% to about 0.08% by weight of the emulsion; and
tretinoin, from about 0.05% to about 1% by weight of the emulsion.

In certain embodiments, the invention relates to an emulsion, comprising:
water, from about 24.00% to about 90.00% by weight of the emulsion;
oleic acid, from about 1.50% to about 14.00% by weight of the emulsion;
diethyl sebacate, from about 2.00% to about 12.00% by weight of the emulsion;
glycerin, from about 2.50% to about 10.00% by weight of the emulsion;
pentylene glycol, from about 1.00% to about 4.00% by weight of the emulsion;
cetostearyl alcohol, from about 0.50% to about 5.00% by weight of the emulsion;
emulsifying wax, from about 0.50% to about 5.00% by weight of the emulsion;
sodium phosphate monobasic, from about 0.50% to about 1.50% by weight of the emulsion;
Polyethylene glycol octadecyl ether, from about 0.20% to about 3.00% by weight of the emulsion;
Poloxamer 188, from about 0.01% to about 2.00% by weight of the emulsion;
methylparaben, from about 0.01% to about 0.50% by weight of the emulsion;
sodium hydroxide, from about 0.01% to about 0.3% by weight of the emulsion;
propylparaben, from about 0.05% to about 0.20% by weight of the emulsion;
disodium EDTA, from about 0.05% to about 0.20% by weight of the emulsion;
butylated hydroxytoluene, from about 0.01% to about 0.08% by weight of the emulsion; and
tretinoin, from about 0.05% to about 1.00% by weight of the emulsion.

In certain embodiments, the invention relates to an emulsion, consisting essentially of:
water, from about 24.00% to about 90.00% by weight of the emulsion;
oleic acid, from about 1.50% to about 14.00% by weight of the emulsion;
diethyl sebacate, from about 2.00% to about 12.00% by weight of the emulsion;
glycerin, from about 2.50% to about 10.00% by weight of the emulsion;
pentylene glycol, from about 1.00% to about 4.00% by weight of the emulsion;
cetostearyl alcohol, from about 0.50% to about 5.00% by weight of the emulsion;
emulsifying wax, from about 0.50% to about 5.00% by weight of the emulsion;
sodium phosphate monobasic, from about 0.50% to about 1.50% by weight of the emulsion;
Polyethylene glycol octadecyl ether, from about 0.20% to about 3.00% by weight of the emulsion;
Poloxamer 188, from about 0.01% to about 2.00% by weight of the emulsion;
methylparaben, from about 0.01% to about 0.50% by weight of the emulsion;
sodium hydroxide, from about 0.01% to about 0.3% by weight of the emulsion;
propylparaben, from about 0.05% to about 0.20% by weight of the emulsion;
disodium EDTA, from about 0.05% to about 0.20% by weight of the emulsion;
butylated hydroxytoluene, from about 0.01% to about 0.08% by weight of the emulsion; and
tretinoin, from about 0.05% to about 1.00% by weight of the emulsion.

In certain embodiments, the invention relates to an emulsion, consisting of:
water, from about 24.00% to about 90.00% by weight of the emulsion;
oleic acid, from about 1.50% to about 14.00% by weight of the emulsion;
diethyl sebacate, from about 2.00% to about 12.00% by weight of the emulsion;
glycerin, from about 2.50% to about 10.00% by weight of the emulsion;
pentylene glycol, from about 1.00% to about 4.00% by weight of the emulsion;
cetostearyl alcohol, from about 0.50% to about 5.00% by weight of the emulsion;
emulsifying wax, from about 0.50% to about 5.00% by weight of the emulsion;
sodium phosphate monobasic, from about 0.50% to about 1.50% by weight of the emulsion;
Polyethylene glycol octadecyl ether, from about 0.20% to about 3.00% by weight of the emulsion;
Poloxamer 188, from about 0.01% to about 2.00% by weight of the emulsion;
methylparaben, from about 0.01% to about 0.50% by weight of the emulsion;
sodium hydroxide, from about 0.01% to about 0.3% by weight of the emulsion;
propylparaben, from about 0.05% to about 0.20% by weight of the emulsion;
disodium EDTA, from about 0.05% to about 0.20% by weight of the emulsion;
butylated hydroxytoluene, from about 0.01% to about 0.08% by weight of the emulsion; and
tretinoin, from about 0.05% to about 1.00% by weight of the emulsion.

In certain embodiments, the invention relates to an emulsion, comprising:
water, from about 24% to about 90% by weight of the emulsion;
oleic acid, from about 1.5% to about 14% by weight of the emulsion;
diethyl sebacate, from about 2% to about 12% by weight of the emulsion;
glycerin, from about 2.5% to about 10% by weight of the emulsion;
pentylene glycol, from about 1% to about 4% by weight of the emulsion;
cetostearyl alcohol, from about 0.5% to about 5% by weight of the emulsion;
emulsifying wax, from about 0.5% to about 5% by weight of the emulsion;
petrolatum, from about 0.5% to about 6% by weight of the emulsion;
Ceteth-20, from about 3% to about 9% by weight of the emulsion;
sodium phosphate monobasic, from about 0.5% to about 1.5% by weight of the emulsion;
Poloxamer 188, from about 0.01% to about 2% by weight of the emulsion;
methylparaben, from about 0.01% to about 0.5% by weight of the emulsion;
sodium hydroxide, from about 0.01% to about 0.3% by weight of the emulsion;
propylparaben, from about 0.05% to about 0.2% by weight of the emulsion;
disodium EDTA, from about 0.05% to about 0.2% by weight of the emulsion;
butylated hydroxytoluene, from about 0.01% to about 0.08% by weight of the emulsion; and
tretinoin, from about 0.05% to about 1% by weight of the emulsion.

In certain embodiments, the invention relates to an emulsion, consisting essentially of:
water, from about 24% to about 90% by weight of the emulsion;
oleic acid, from about 1.5% to about 14% by weight of the emulsion;
diethyl sebacate, from about 2% to about 12% by weight of the emulsion;
glycerin, from about 2.5% to about 10% by weight of the emulsion;
pentylene glycol, from about 1% to about 4% by weight of the emulsion;
cetostearyl alcohol, from about 0.5% to about 5% by weight of the emulsion;
emulsifying wax, from about 0.5% to about 5% by weight of the emulsion;
petrolatum, from about 0.5% to about 6% by weight of the emulsion;
Ceteth-20, from about 3% to about 9% by weight of the emulsion;
sodium phosphate monobasic, from about 0.5% to about 1.5% by weight of the emulsion;
Poloxamer 188, from about 0.01% to about 2% by weight of the emulsion;
methylparaben, from about 0.01% to about 0.5% by weight of the emulsion;
sodium hydroxide, from about 0.01% to about 0.3% by weight of the emulsion;
propylparaben, from about 0.05% to about 0.2% by weight of the emulsion;
disodium EDTA, from about 0.05% to about 0.2% by weight of the emulsion;
butylated hydroxytoluene, from about 0.01% to about 0.08% by weight of the emulsion; and
tretinoin, from about 0.05% to about 1% by weight of the emulsion.

In certain embodiments, the invention relates to an emulsion, consisting of:
water, from about 24% to about 90% by weight of the emulsion;
oleic acid, from about 1.5% to about 14% by weight of the emulsion;
diethyl sebacate, from about 2% to about 12% by weight of the emulsion;
glycerin, from about 2.5% to about 10% by weight of the emulsion;
pentylene glycol, from about 1% to about 4% by weight of the emulsion;
cetostearyl alcohol, from about 0.5% to about 5% by weight of the emulsion;
emulsifying wax, from about 0.5% to about 5% by weight of the emulsion;
petrolatum, from about 0.5% to about 6% by weight of the emulsion;
Ceteth-20, from about 3% to about 9% by weight of the emulsion;
sodium phosphate monobasic, from about 0.5% to about 1.5% by weight of the emulsion;
Poloxamer 188, from about 0.01% to about 2% by weight of the emulsion;
methylparaben, from about 0.01% to about 0.5% by weight of the emulsion;
sodium hydroxide, from about 0.01% to about 0.3% by weight of the emulsion;
propylparaben, from about 0.05% to about 0.2% by weight of the emulsion;
disodium EDTA, from about 0.05% to about 0.2% by weight of the emulsion;
butylated hydroxytoluene, from about 0.01% to about 0.08% by weight of the emulsion; and
tretinoin, from about 0.05% to about 1% by weight of the emulsion.

In certain embodiments, the invention relates to an emulsion, comprising:
water, from about 24.00% to about 90.00% by weight of the emulsion;
oleic acid, from about 1.50% to about 14.00% by weight of the emulsion;
diethyl sebacate, from about 2.00% to about 12.00% by weight of the emulsion;
glycerin, from about 2.50% to about 10.00% by weight of the emulsion;
pentylene glycol, from about 1.00% to about 4.00% by weight of the emulsion;
cetostearyl alcohol, from about 0.50% to about 5.00% by weight of the emulsion;
emulsifying wax, from about 0.50% to about 5.00% by weight of the emulsion;
petrolatum, from about 0.50% to about 6.00% by weight of the emulsion;
Ceteth-20, from about 3.00% to about 9.00% by weight of the emulsion;
sodium phosphate monobasic, from about 0.50% to about 1.50% by weight of the emulsion;
Poloxamer 188, from about 0.01% to about 2.00% by weight of the emulsion;
methylparaben, from about 0.01% to about 0.50% by weight of the emulsion;
sodium hydroxide, from about 0.01% to about 0.30% by weight of the emulsion;
propylparaben, from about 0.05% to about 0.20% by weight of the emulsion;
disodium EDTA, from about 0.05% to about 0.20% by weight of the emulsion;
butylated hydroxytoluene, from about 0.01% to about 0.08% by weight of the emulsion; and
tretinoin, from about 0.05% to about 1.00% by weight of the emulsion.

In certain embodiments, the invention relates to an emulsion, consisting essentially of:
water, from about 24.00% to about 90.00% by weight of the emulsion;
oleic acid, from about 1.50% to about 14.00% by weight of the emulsion;
diethyl sebacate, from about 2.00% to about 12.00% by weight of the emulsion;
glycerin, from about 2.50% to about 10.00% by weight of the emulsion;
pentylene glycol, from about 1.00% to about 4.00% by weight of the emulsion;
cetostearyl alcohol, from about 0.50% to about 5.00% by weight of the emulsion;
emulsifying wax, from about 0.50% to about 5.00% by weight of the emulsion;
petrolatum, from about 0.50% to about 6.00% by weight of the emulsion;
Ceteth-20, from about 3.00% to about 9.00% by weight of the emulsion;
sodium phosphate monobasic, from about 0.50% to about 1.50% by weight of the emulsion;
Poloxamer 188, from about 0.01% to about 2.00% by weight of the emulsion;
methylparaben, from about 0.01% to about 0.50% by weight of the emulsion;
sodium hydroxide, from about 0.01% to about 0.30% by weight of the emulsion;
propylparaben, from about 0.05% to about 0.20% by weight of the emulsion;
disodium EDTA, from about 0.05% to about 0.20% by weight of the emulsion;
butylated hydroxytoluene, from about 0.01% to about 0.08% by weight of the emulsion; and
tretinoin, from about 0.05% to about 1.00% by weight of the emulsion.

In certain embodiments, the invention relates to an emulsion, consisting of:
water, from about 24.00% to about 90.00% by weight of the emulsion;
oleic acid, from about 1.50% to about 14.00% by weight of the emulsion;
diethyl sebacate, from about 2.00% to about 12.00% by weight of the emulsion;
glycerin, from about 2.50% to about 10.00% by weight of the emulsion;
pentylene glycol, from about 1.00% to about 4.00% by weight of the emulsion;
cetostearyl alcohol, from about 0.50% to about 5.00% by weight of the emulsion;
emulsifying wax, from about 0.50% to about 5.00% by weight of the emulsion;
petrolatum, from about 0.50% to about 6.00% by weight of the emulsion;
Ceteth-20, from about 3.00% to about 9.00% by weight of the emulsion;
sodium phosphate monobasic, from about 0.50% to about 1.50% by weight of the emulsion;
Poloxamer 188, from about 0.01% to about 2.00% by weight of the emulsion;
methylparaben, from about 0.01% to about 0.50% by weight of the emulsion;
sodium hydroxide, from about 0.01% to about 0.30% by weight of the emulsion;
propylparaben, from about 0.05% to about 0.20% by weight of the emulsion;
disodium EDTA, from about 0.05% to about 0.20% by weight of the emulsion;
butylated hydroxytoluene, from about 0.01% to about 0.08% by weight of the emulsion; and
tretinoin, from about 0.05% to about 1.00% by weight of the emulsion.

In certain embodiments, the invention relates to an emulsion, comprising:
water, from about 24% to about 90% by weight of the emulsion;
diethyl sebacate, from about 2% to about 12% by weight of the emulsion;
glycerin, from about 2.5% to about 10% by weight of the emulsion;
isostearyl alcohol, from about 2% to about 6% by weight of the emulsion;
pentylene glycol, from about 1% to about 4% by weight of the emulsion;
cetostearyl alcohol, from about 0.5% to about 5% by weight of the emulsion;
emulsifying wax, from about 0.5% to about 5% by weight of the emulsion;
petrolatum, from about 0.5% to about 6% by weight of the emulsion;
sodium phosphate monobasic, from about 0.5% to about 1.5% by weight of the emulsion;
Poloxamer 188, from about 0.01% to about 2% by weight of the emulsion;
methylparaben, from about 0.01% to about 0.5% by weight of the emulsion;
sodium hydroxide, from about 0.01% to about 0.3% by weight of the emulsion;
propylparaben, from about 0.05% to about 0.2% by weight of the emulsion;
disodium EDTA, from about 0.05% to about 0.2% by weight of the emulsion;
butylated hydroxytoluene, from about 0.01% to about 0.08% by weight of the emulsion; and
tretinoin, from about 0.05% to about 1% by weight of the emulsion.

In certain embodiments, the invention relates to an emulsion, consisting essentially of:
water, from about 24% to about 90% by weight of the emulsion;
diethyl sebacate, from about 2% to about 12% by weight of the emulsion;
glycerin, from about 2.5% to about 10% by weight of the emulsion;
isostearyl alcohol, from about 2% to about 6% by weight of the emulsion;
pentylene glycol, from about 1% to about 4% by weight of the emulsion;
cetostearyl alcohol, from about 0.5% to about 5% by weight of the emulsion;
emulsifying wax, from about 0.5% to about 5% by weight of the emulsion;
petrolatum, from about 0.5% to about 6% by weight of the emulsion;
sodium phosphate monobasic, from about 0.5% to about 1.5% by weight of the emulsion;
Poloxamer 188, from about 0.01% to about 2% by weight of the emulsion;
methylparaben, from about 0.01% to about 0.5% by weight of the emulsion;
sodium hydroxide, from about 0.01% to about 0.3% by weight of the emulsion;
propylparaben, from about 0.05% to about 0.2% by weight of the emulsion;
disodium EDTA, from about 0.05% to about 0.2% by weight of the emulsion;
butylated hydroxytoluene, from about 0.01% to about 0.08% by weight of the emulsion; and
tretinoin, from about 0.05% to about 1% by weight of the emulsion.

In certain embodiments, the invention relates to an emulsion, consisting of:
water, from about 24% to about 90% by weight of the emulsion;
diethyl sebacate, from about 2% to about 12% by weight of the emulsion;
glycerin, from about 2.5% to about 10% by weight of the emulsion;
isostearyl alcohol, from about 2% to about 6% by weight of the emulsion;
pentylene glycol, from about 1% to about 4% by weight of the emulsion;
cetostearyl alcohol, from about 0.5% to about 5% by weight of the emulsion;
emulsifying wax, from about 0.5% to about 5% by weight of the emulsion;
petrolatum, from about 0.5% to about 6% by weight of the emulsion;
sodium phosphate monobasic, from about 0.5% to about 1.5% by weight of the emulsion;
Poloxamer 188, from about 0.01% to about 2% by weight of the emulsion;
methylparaben, from about 0.01% to about 0.5% by weight of the emulsion;
sodium hydroxide, from about 0.01% to about 0.3% by weight of the emulsion;
propylparaben, from about 0.05% to about 0.2% by weight of the emulsion;
disodium EDTA, from about 0.05% to about 0.2% by weight of the emulsion;
butylated hydroxytoluene, from about 0.01% to about 0.08% by weight of the emulsion; and
tretinoin, from about 0.05% to about 1% by weight of the emulsion.

In certain embodiments, the invention relates to an emulsion, comprising:
water, from about 24.00% to about 90.00% by weight of the emulsion;
diethyl sebacate, from about 2.00% to about 12.00% by weight of the emulsion;
glycerin, from about 2.50% to about 10.00% by weight of the emulsion;
isostearyl alcohol, from about 2.00% to about 6.00% by weight of the emulsion;
pentylene glycol, from about 1.00% to about 4.00% by weight of the emulsion;
cetostearyl alcohol, from about 0.50% to about 5.00% by weight of the emulsion;
emulsifying wax, from about 0.50% to about 5.00% by weight of the emulsion;
petrolatum, from about 0.50% to about 6.00% by weight of the emulsion;
sodium phosphate monobasic, from about 0.50% to about 1.50% by weight of the emulsion;
Poloxamer 188, from about 0.01% to about 2.00% by weight of the emulsion;
methylparaben, from about 0.01% to about 0.50% by weight of the emulsion;
sodium hydroxide, from about 0.01% to about 0.3% by weight of the emulsion;
propylparaben, from about 0.05% to about 0.20% by weight of the emulsion;
disodium EDTA, from about 0.05% to about 0.20% by weight of the emulsion;
butylated hydroxytoluene, from about 0.01% to about 0.08% by weight of the emulsion; and
tretinoin, from about 0.05% to 1.00% by weight of the emulsion.

In certain embodiments, the invention relates to an emulsion, consisting essentially of:
water, from about 24.00% to about 90.00% by weight of the emulsion;
diethyl sebacate, from about 2.00% to about 12.00% by weight of the emulsion;
glycerin, from about 2.50% to about 10.00% by weight of the emulsion;
isostearyl alcohol, from about 2.00% to about 6.00% by weight of the emulsion;
pentylene glycol, from about 1.00% to about 4.00% by weight of the emulsion;
cetostearyl alcohol, from about 0.50% to about 5.00% by weight of the emulsion;
emulsifying wax, from about 0.50% to about 5.00% by weight of the emulsion;
petrolatum, from about 0.50% to about 6.00% by weight of the emulsion;
sodium phosphate monobasic, from about 0.50% to about 1.50% by weight of the emulsion;
Poloxamer 188, from about 0.01% to about 2.00% by weight of the emulsion;
methylparaben, from about 0.01% to about 0.50% by weight of the emulsion;
sodium hydroxide, from about 0.01% to about 0.3% by weight of the emulsion;
propylparaben, from about 0.05% to about 0.20% by weight of the emulsion;
disodium EDTA, from about 0.05% to about 0.20% by weight of the emulsion;
butylated hydroxytoluene, from about 0.01% to about 0.08% by weight of the emulsion; and
tretinoin, from about 0.05% to about 1.00% by weight of the emulsion.

In certain embodiments, the invention relates to an emulsion, consisting of:
water, from about 24.00% to about 90.00% by weight of the emulsion;
diethyl sebacate, from about 2.00% to about 12.00% by weight of the emulsion;
glycerin, from about 2.50% to about 10.00% by weight of the emulsion;
isostearyl alcohol, from about 2.00% to about 6.00% by weight of the emulsion;
pentylene glycol, from about 1.00% to about 4.00% by weight of the emulsion;
cetostearyl alcohol, from about 0.50% to about 5.00% by weight of the emulsion;
emulsifying wax, from about 0.50% to about 5.00% by weight of the emulsion;
petrolatum, from about 0.50% to about 6.00% by weight of the emulsion;
sodium phosphate monobasic, from about 0.50% to about 1.50% by weight of the emulsion;
Poloxamer 188, from about 0.01% to about 2.00% by weight of the emulsion;
methylparaben, from about 0.01% to about 0.50% by weight of the emulsion;
sodium hydroxide, from about 0.01% to about 0.3% by weight of the emulsion;
propylparaben, from about 0.05% to about 0.20% by weight of the emulsion;
disodium EDTA, from about 0.05% to about 0.20% by weight of the emulsion;
butylated hydroxytoluene, from about 0.01% to about 0.08% by weight of the emulsion; and
tretinoin, from about 0.05% to about 1.00% by weight of the emulsion.

In certain embodiments, the invention relates to an emulsion, comprising:
water, in about 71% by weight of the emulsion;
sodium hyaluronate, in about 0.1% by weight of the emulsion;
glycerin, in about 7.5% by weight of the emulsion;
ethylhexyl palmitate, in about 6% by weight of the emulsion;
cetostearyl alcohol, in about 3% by weight of the emulsion;
dicetyl phosphate, in about 2% by weight of the emulsion;
ceteth-10 phosphate, in about 1% by weight of the emulsion;
propylene glycol, in about 2.5% by weight of the emulsion;
theobroma grandiflorum seed butter, in about 2% by weight of the emulsion;
dimethicone, in about 1% by weight of the emulsion;
petrolatum, in about 1% by weight of the emulsion;
Polyethylene glycol octadecyl ether, in about 0.86% by weight of the emulsion;
tocopheryl acetate, in about 0.5% by weight of the emulsion;
1,3-bis(N-2-(hydroxyethyl)palmitoylamino)-2-hydroxypropane, in about 0.5% by weight of the emulsion;
methylparaben, in about 0.3% by weight of the emulsion;
sodium hydroxide, in about 0.2% by weight of the emulsion;
propylparaben, in about 0.1% by weight of the emulsion; and
disodium EDTA, in about 0.1% by weight of the emulsion.

In certain embodiments, the invention relates to an emulsion, consisting essentially of:
water, in about 71% by weight of the emulsion;
sodium hyaluronate, in about 0.1% by weight of the emulsion;
glycerin, in about 7.5% by weight of the emulsion;
ethylhexyl palmitate, in about 6% by weight of the emulsion;
cetostearyl alcohol, in about 3% by weight of the emulsion;
dicetyl phosphate, in about 2% by weight of the emulsion;
ceteth-10 phosphate, in about 1% by weight of the emulsion;
propylene glycol, in about 2.5% by weight of the emulsion;
theobroma grandiflorum seed butter, in about 2% by weight of the emulsion;
dimethicone, in about 1% by weight of the emulsion;
petrolatum, in about 1% by weight of the emulsion;
Polyethylene glycol octadecyl ether, in about 0.86% by weight of the emulsion;
tocopheryl acetate, in about 0.5% by weight of the emulsion;
1,3-bis(N-2-(hydroxyethyl)palmitoylamino)-2-hydroxypropane, in about 0.5% by weight of the emulsion;
methylparaben, in about 0.3% by weight of the emulsion;
sodium hydroxide, in about 0.2% by weight of the emulsion;
propylparaben, in about 0.1% by weight of the emulsion; and
disodium EDTA, in about 0.1% by weight of the emulsion.

In certain embodiments, the invention relates to an emulsion, consisting of:
water, in about 71% by weight of the emulsion;
sodium hyaluronate, in about 0.1% by weight of the emulsion;
glycerin, in about 7.5% by weight of the emulsion;
ethylhexyl palmitate, in about 6% by weight of the emulsion;
cetostearyl alcohol, in about 3% by weight of the emulsion;
dicetyl phosphate, in about 2% by weight of the emulsion;
ceteth-10 phosphate, in about 1% by weight of the emulsion;
propylene glycol, in about 2.5% by weight of the emulsion;
theobroma grandiflorum seed butter, in about 2% by weight of the emulsion;
dimethicone, in about 1% by weight of the emulsion;
petrolatum, in about 1% by weight of the emulsion;
Polyethylene glycol octadecyl ether, in about 0.86% by weight of the emulsion;
tocopheryl acetate, in about 0.5% by weight of the emulsion;
1,3-bis(N-2-(hydroxyethyl)palmitoylamino)-2-hydroxypropane, in about 0.5% by weight of the emulsion;
methylparaben, in about 0.3% by weight of the emulsion;
sodium hydroxide, in about 0.2% by weight of the emulsion;
propylparaben, in about 0.1% by weight of the emulsion; and
disodium EDTA, in about 0.1% by weight of the emulsion.

In certain embodiments, the invention relates to an emulsion, comprising:
water, in about 71.34% by weight of the emulsion;
sodium hyaluronate, in about 0.10% by weight of the emulsion;
glycerin, in about 7.50% by weight of the emulsion;
ethylhexyl palmitate, in about 6.00% by weight of the emulsion;
cetostearyl alcohol, in about 3.00% by weight of the emulsion;
dicetyl phosphate, in about 2.10% by weight of the emulsion;
ceteth-10 phosphate, in about 0.90% by weight of the emulsion;
propylene glycol, in about 2.50% by weight of the emulsion;
theobroma grandiflorum seed butter, in about 2.00% by weight of the emulsion;
dimethicone, in about 1.00% by weight of the emulsion;
petrolatum, in about 1.00% by weight of the emulsion;
Polyethylene glycol octadecyl ether, in about 0.86% by weight of the emulsion;
tocopheryl acetate, in about 0.50% by weight of the emulsion;
1,3-bis(N-2-(hydroxyethyl)palmitoylamino)-2-hydroxypropane, in about 0.50% by weight of the emulsion;
methylparaben, in about 0.30% by weight of the emulsion;
sodium hydroxide, in about 0.20% by weight of the emulsion;
propylparaben, in about 0.10% by weight of the emulsion; and
disodium EDTA, in about 0.10% by weight of the emulsion.

In certain embodiments, the invention relates to an emulsion, consisting essentially of:
water, in about 71.34% by weight of the emulsion;
sodium hyaluronate, in about 0.10% by weight of the emulsion;
glycerin, in about 7.50% by weight of the emulsion;
ethylhexyl palmitate, in about 6.00% by weight of the emulsion;
cetostearyl alcohol, in about 3.00% by weight of the emulsion;
dicetyl phosphate, in about 2.10% by weight of the emulsion;
ceteth-10 phosphate, in about 0.90% by weight of the emulsion;
propylene glycol, in about 2.50% by weight of the emulsion;
theobroma grandiflorum seed butter, in about 2.00% by weight of the emulsion;
dimethicone, in about 1.00% by weight of the emulsion;
petrolatum, in about 1.00% by weight of the emulsion;
Polyethylene glycol octadecyl ether, in about 0.86% by weight of the emulsion;
tocopheryl acetate, in about 0.50% by weight of the emulsion;
1,3-bis(N-2-(hydroxyethyl)palmitoylamino)-2-hydroxypropane, in about 0.50% by weight of the emulsion;
methylparaben, in about 0.30% by weight of the emulsion;
sodium hydroxide, in about 0.20% by weight of the emulsion;
propylparaben, in about 0.10% by weight of the emulsion; and
disodium EDTA, in about 0.10% by weight of the emulsion.

In certain embodiments, the invention relates to an emulsion, consisting of:
water, in about 71.34% by weight of the emulsion;
sodium hyaluronate, in about 0.10% by weight of the emulsion;
glycerin, in about 7.50% by weight of the emulsion;
ethylhexyl palmitate, in about 6.00% by weight of the emulsion;
cetostearyl alcohol, in about 3.00% by weight of the emulsion;
dicetyl phosphate, in about 2.10% by weight of the emulsion;
ceteth-10 phosphate, in about 0.90% by weight of the emulsion;
propylene glycol, in about 2.50% by weight of the emulsion;
theobroma grandiflorum seed butter, in about 2.00% by weight of the emulsion;
dimethicone, in about 1.00% by weight of the emulsion;
petrolatum, in about 1.00% by weight of the emulsion;
Polyethylene glycol octadecyl ether, in about 0.86% by weight of the emulsion;
tocopheryl acetate, in about 0.50% by weight of the emulsion;
1,3-bis(N-2-(hydroxyethyl)palmitoylamino)-2-hydroxypropane, in about 0.50% by weight of the emulsion;
methylparaben, in about 0.30% by weight of the emulsion;
sodium hydroxide, in about 0.20% by weight of the emulsion;
propylparaben, in about 0.10% by weight of the emulsion; and
disodium EDTA, in about 0.10% by weight of the emulsion.

In certain embodiments, the invention relates to an emulsion, comprising:

water, in about 78% by weight of the emulsion;

benzoyl peroxide, in about 9.8% by weight of the emulsion;

glycerin, in about 5% by weight of the emulsion;

propylene glycol, in about 2.5% by weight of the emulsion;

cetostearyl alcohol, in about 1.1% by weight of the emulsion;

emulsifying wax, in about 1.1% by weight of the emulsion;

dimethicone, in about 0.45% by weight of the emulsion;

sodium citrate, in about 0.62% by weight of the emulsion;

Polyethylene glycol octadecyl ether, in about 0.45% by weight of the emulsion;

methylparaben, in about 0.3% by weight of the emulsion;

C₁₂-C₁₅ alkyl benzoates, in about 0.22% by weight of the emulsion;

propylparaben, in about 0.1% by weight of the emulsion;

disodium EDTA, in about 0.1% by weight of the emulsion;

butylated hydroxytoluene, in about 0.05% by weight of the emulsion; and

citric acid, in about 0.05% by weight of the emulsion.

In certain embodiments, the invention relates to an emulsion, consisting essentially of:

water, in about 78% by weight of the emulsion;

benzoyl peroxide, in about 9.8% by weight of the emulsion;

glycerin, in about 5% by weight of the emulsion;

propylene glycol, in about 2.5% by weight of the emulsion;

cetostearyl alcohol, in about 1.1% by weight of the emulsion;

emulsifying wax, in about 1.1% by weight of the emulsion;

dimethicone, in about 0.45% by weight of the emulsion;

sodium citrate, in about 0.62% by weight of the emulsion;

Polyethylene glycol octadecyl ether, in about 0.45% by weight of the emulsion;

methylparaben, in about 0.3% by weight of the emulsion;

C₁₂-C₁₅ alkyl benzoates, in about 0.22% by weight of the emulsion;

propylparaben, in about 0.1% by weight of the emulsion;

disodium EDTA, in about 0.1% by weight of the emulsion;

butylated hydroxytoluene, in about 0.05% by weight of the emulsion; and

citric acid, in about 0.05% by weight of the emulsion.

In certain embodiments, the invention relates to an emulsion, consisting of:

water, in about 78% by weight of the emulsion;

benzoyl peroxide, in about 9.8% by weight of the emulsion;

glycerin, in about 5% by weight of the emulsion;

propylene glycol, in about 2.5% by weight of the emulsion;

cetostearyl alcohol, in about 1.1% by weight of the emulsion;

emulsifying wax, in about 1.1% by weight of the emulsion;

dimethicone, in about 0.45% by weight of the emulsion;

sodium citrate, in about 0.62% by weight of the emulsion;

Polyethylene glycol octadecyl ether, in about 0.45% by weight of the emulsion;

methylparaben, in about 0.3% by weight of the emulsion;

C₁₂-C₁₅ alkyl benzoates, in about 0.22% by weight of the emulsion;

propylparaben, in about 0.1% by weight of the emulsion;

disodium EDTA, in about 0.1% by weight of the emulsion;

butylated hydroxytoluene, in about 0.05% by weight of the emulsion; and

citric acid, in about 0.05% by weight of the emulsion.

In certain embodiments, the invention relates to an emulsion, comprising:

water, in about 78.16% by weight of the emulsion;

benzoyl peroxide, in about 9.80% by weight of the emulsion;

glycerin, in about 5.00% by weight of the emulsion;

propylene glycol, in about 2.50% by weight of the emulsion;

cetostearyl alcohol, in about 1.10% by weight of the emulsion;

emulsifying wax, in about 1.10% by weight of the emulsion;

dimethicone, in about 0.45% by weight of the emulsion;

sodium citrate, in about 0.62% by weight of the emulsion;

Polyethylene glycol octadecyl ether, in about 0.45% by weight of the emulsion;

methylparaben, in about 0.30% by weight of the emulsion;

C₁₂-C₁₅ alkyl benzoates, in about 0.22% by weight of the emulsion;

propylparaben, in about 0.10% by weight of the emulsion;

disodium EDTA, in about 0.10% by weight of the emulsion;

butylated hydroxytoluene, in about 0.05% by weight of the emulsion; and

citric acid, in about 0.05% by weight of the emulsion.

In certain embodiments, the invention relates to an emulsion, consisting essentially of:

water, in about 78.16% by weight of the emulsion;

benzoyl peroxide, in about 9.80% by weight of the emulsion;

glycerin, in about 5.00% by weight of the emulsion;

propylene glycol, in about 2.50% by weight of the emulsion;

cetostearyl alcohol, in about 1.10% by weight of the emulsion;

emulsifying wax, in about 1.10% by weight of the emulsion;

dimethicone, in about 0.45% by weight of the emulsion;

sodium citrate, in about 0.62% by weight of the emulsion;

Polyethylene glycol octadecyl ether, in about 0.45% by weight of the emulsion;

methylparaben, in about 0.30% by weight of the emulsion;

C₁₂-C₁₅ alkyl benzoates, in about 0.22% by weight of the emulsion;

propylparaben, in about 0.10% by weight of the emulsion;

disodium EDTA, in about 0.10% by weight of the emulsion;

butylated hydroxytoluene, in about 0.05% by weight of the emulsion; and

citric acid, in about 0.05% by weight of the emulsion.

In certain embodiments, the invention relates to an emulsion, consisting of:

water, in about 78.16% by weight of the emulsion;

benzoyl peroxide, in about 9.80% by weight of the emulsion;

glycerin, in about 5.00% by weight of the emulsion;

propylene glycol, in about 2.50% by weight of the emulsion;

cetostearyl alcohol, in about 1.10% by weight of the emulsion;

emulsifying wax, in about 1.10% by weight of the emulsion;

dimethicone, in about 0.45% by weight of the emulsion;

sodium citrate, in about 0.62% by weight of the emulsion;

Polyethylene glycol octadecyl ether, in about 0.45% by weight of the emulsion;

methylparaben, in about 0.30% by weight of the emulsion;

C₁₂-C₁₅ alkyl benzoates, in about 0.22% by weight of the emulsion;

propylparaben, in about 0.10% by weight of the emulsion;

disodium EDTA, in about 0.10% by weight of the emulsion;

butylated hydroxytoluene, in about 0.05% by weight of the emulsion; and

citric acid, in about 0.05% by weight of the emulsion.

In certain embodiments, the invention relates to an emulsion, comprising:

water, in about 69% by weight of the emulsion;

oleic acid, in about 9% by weight of the emulsion;

glycerin, in about 7% by weight of the emulsion;

alfa terpineol, in about 3% by weight of the emulsion;

pentylene glycol, in about 2.5% by weight of the emulsion;

cetostearyl alcohol, in about 3% by weight of the emulsion;

emulsifying wax, in about 3% by weight of the emulsion;

sodium phosphate monobasic, in about 1% by weight of the emulsion;

Polyethylene glycol octadecyl ether, in about 2% by weight of the emulsion;

Poloxamer 188, in about 0.3% by weight of the emulsion;

methylparaben, in about 0.3% by weight of the emulsion;

sodium hydroxide, in about 0.03% by weight of the emulsion;

propylparaben, in about 0.1% by weight of the emulsion;

disodium EDTA, in about 0.1% by weight of the emulsion;

butylated hydroxytoluene, in about 0.03% by weight of the emulsion; and

tretinoin, in about 0.1% by weight of the emulsion.

In certain embodiments, the invention relates to an emulsion, consisting essentially of:

water, in about 69% by weight of the emulsion;

oleic acid, in about 9% by weight of the emulsion;

glycerin, in about 7% by weight of the emulsion;

alfa terpineol, in about 3% by weight of the emulsion;

pentylene glycol, in about 2.5% by weight of the emulsion;

cetostearyl alcohol, in about 3% by weight of the emulsion;

emulsifying wax, in about 3% by weight of the emulsion;

sodium phosphate monobasic, in about 1% by weight of the emulsion;

Polyethylene glycol octadecyl ether, in about 2% by weight of the emulsion;

Poloxamer 188, in about 0.3% by weight of the emulsion;

methylparaben, in about 0.3% by weight of the emulsion;

sodium hydroxide, in about 0.03% by weight of the emulsion;

propylparaben, in about 0.1% by weight of the emulsion;

disodium EDTA, in about 0.1% by weight of the emulsion;

butylated hydroxytoluene, in about 0.03% by weight of the emulsion; and

tretinoin, in about 0.1% by weight of the emulsion.

In certain embodiments, the invention relates to an emulsion, consisting of:

water, in about 69% by weight of the emulsion;

oleic acid, in about 9% by weight of the emulsion;

glycerin, in about 7% by weight of the emulsion;

alfa terpineol, in about 3% by weight of the emulsion;

pentylene glycol, in about 2.5% by weight of the emulsion;

cetostearyl alcohol, in about 3% by weight of the emulsion;

emulsifying wax, in about 3% by weight of the emulsion;

sodium phosphate monobasic, in about 1% by weight of the emulsion;

Polyethylene glycol octadecyl ether, in about 2% by weight of the emulsion;

Poloxamer 188, in about 0.3% by weight of the emulsion;

methylparaben, in about 0.3% by weight of the emulsion;

sodium hydroxide, in about 0.03% by weight of the emulsion;

propylparaben, in about 0.1% by weight of the emulsion;

disodium EDTA, in about 0.1% by weight of the emulsion;

butylated hydroxytoluene, in about 0.03% by weight of the emulsion; and

tretinoin, in about 0.1% by weight of the emulsion.

In certain embodiments, the invention relates to an emulsion, comprising:

water, in about 68.54% by weight of the emulsion;

oleic acid, in about 9.00% by weight of the emulsion;

glycerin, in about 7.00% by weight of the emulsion;

alfa terpineol, in about 3.00% by weight of the emulsion;

pentylene glycol, in about 2.50% by weight of the emulsion;

cetostearyl alcohol, in about 3.00% by weight of the emulsion;

emulsifying wax, in about 3.00% by weight of the emulsion;

sodium phosphate monobasic, in about 1.00% by weight of the emulsion;

Polyethylene glycol octadecyl ether, in about 2.00% by weight of the emulsion;

Poloxamer 188, in about 0.30% by weight of the emulsion;

methylparaben, in about 0.30% by weight of the emulsion;

sodium hydroxide, in about 0.03% by weight of the emulsion;

propylparaben, in about 0.10% by weight of the emulsion;

disodium EDTA, in about 0.10% by weight of the emulsion;

butylated hydroxytoluene, in about 0.03% by weight of the emulsion; and

tretinoin, in about 0.10% by weight of the emulsion.

In certain embodiments, the invention relates to an emulsion, consisting essentially of:

water, in about 68.54% by weight of the emulsion;

oleic acid, in about 9.00% by weight of the emulsion;

glycerin, in about 7.00% by weight of the emulsion;

alfa terpineol, in about 3.00% by weight of the emulsion;

pentylene glycol, in about 2.50% by weight of the emulsion;

cetostearyl alcohol, in about 3.00% by weight of the emulsion;

emulsifying wax, in about 3.00% by weight of the emulsion;

sodium phosphate monobasic, in about 1.00% by weight of the emulsion;

Polyethylene glycol octadecyl ether, in about 2.00% by weight of the emulsion;

Poloxamer 188, in about 0.30% by weight of the emulsion;

methylparaben, in about 0.30% by weight of the emulsion;

sodium hydroxide, in about 0.03% by weight of the emulsion;

propylparaben, in about 0.10% by weight of the emulsion;

disodium EDTA, in about 0.10% by weight of the emulsion;

butylated hydroxytoluene, in about 0.03% by weight of the emulsion; and

tretinoin, in about 0.10% by weight of the emulsion.

In certain embodiments, the invention relates to an emulsion, consisting of:

water, in about 68.54% by weight of the emulsion;

oleic acid, in about 9.00% by weight of the emulsion;

glycerin, in about 7.00% by weight of the emulsion;

alfa terpineol, in about 3.00% by weight of the emulsion;

pentylene glycol, in about 2.50% by weight of the emulsion;

cetostearyl alcohol, in about 3.00% by weight of the emulsion;

emulsifying wax, in about 3.00% by weight of the emulsion;

sodium phosphate monobasic, in about 1.00% by weight of the emulsion;

Polyethylene glycol octadecyl ether, in about 2.00% by weight of the emulsion;

Poloxamer 188, in about 0.30% by weight of the emulsion;

methylparaben, in about 0.30% by weight of the emulsion;

sodium hydroxide, in about 0.03% by weight of the emulsion;

propylparaben, in about 0.10% by weight of the emulsion;

disodium EDTA, in about 0.10% by weight of the emulsion;

butylated hydroxytoluene, in about 0.03% by weight of the emulsion; and

tretinoin, in about 0.10% by weight of the emulsion.

In certain embodiments, the invention relates to an emulsion, comprising:

water, in about 70% by weight of the emulsion;

oleic acid, in about 8% by weight of the emulsion;

diethyl sebacate, in about 4% by weight of the emulsion;

glycerin, in about 7% by weight of the emulsion;

pentylene glycol, in about 2.5% by weight of the emulsion;

cetostearyl alcohol, in about 3% by weight of the emulsion;

emulsifying wax, in about 3% by weight of the emulsion;

sodium phosphate monobasic, in about 1% by weight of the emulsion;

Polyethylene glycol octadecyl ether, in about 1% by weight of the emulsion;

Poloxamer 188, in about 0.3% by weight of the emulsion;

methylparaben, in about 0.3% by weight of the emulsion;

sodium hydroxide, in about 0.03% by weight of the emulsion;

propylparaben, in about 0.1% by weight of the emulsion;

disodium EDTA, in about 0.1% by weight of the emulsion;

butylated hydroxytoluene, in about 0.03% by weight of the emulsion; and

tretinoin, in about 0.1% by weight of the emulsion.

In certain embodiments, the invention relates to an emulsion, consisting essentially of:

water, in about 70% by weight of the emulsion;

oleic acid, in about 8% by weight of the emulsion;

diethyl sebacate, in about 4% by weight of the emulsion;

glycerin, in about 7% by weight of the emulsion;

pentylene glycol, in about 2.5% by weight of the emulsion;

cetostearyl alcohol, in about 3% by weight of the emulsion;

emulsifying wax, in about 3% by weight of the emulsion;

sodium phosphate monobasic, in about 1% by weight of the emulsion;

Polyethylene glycol octadecyl ether, in about 1% by weight of the emulsion;

Poloxamer 188, in about 0.3% by weight of the emulsion;

methylparaben, in about 0.3% by weight of the emulsion;

sodium hydroxide, in about 0.03% by weight of the emulsion;

propylparaben, in about 0.1% by weight of the emulsion;

disodium EDTA, in about 0.1% by weight of the emulsion;

butylated hydroxytoluene, in about 0.03% by weight of the emulsion; and

tretinoin, in about 0.1% by weight of the emulsion.

In certain embodiments, the invention relates to an emulsion, consisting of:

water, in about 70% by weight of the emulsion;

oleic acid, in about 8% by weight of the emulsion;

diethyl sebacate, in about 4% by weight of the emulsion;

glycerin, in about 7% by weight of the emulsion;

pentylene glycol, in about 2.5% by weight of the emulsion;

cetostearyl alcohol, in about 3% by weight of the emulsion;

emulsifying wax, in about 3% by weight of the emulsion;

sodium phosphate monobasic, in about 1% by weight of the emulsion;

Polyethylene glycol octadecyl ether, in about 1% by weight of the emulsion;

Poloxamer 188, in about 0.3% by weight of the emulsion;

methylparaben, in about 0.3% by weight of the emulsion;

sodium hydroxide, in about 0.03% by weight of the emulsion;

propylparaben, in about 0.1% by weight of the emulsion;

disodium EDTA, in about 0.1% by weight of the emulsion;

butylated hydroxytoluene, in about 0.03% by weight of the emulsion; and

tretinoin, in about 0.1% by weight of the emulsion.

In certain embodiments, the invention relates to an emulsion, comprising:

water, in about 69.54% by weight of the emulsion;

oleic acid, in about 8.00% by weight of the emulsion;

diethyl sebacate, in about 4.00% by weight of the emulsion;

glycerin, in about 7.00% by weight of the emulsion;

pentylene glycol, in about 2.50% by weight of the emulsion;

cetostearyl alcohol, in about 3.00% by weight of the emulsion;

emulsifying wax, in about 3.00% by weight of the emulsion;

sodium phosphate monobasic, in about 1.00% by weight of the emulsion;

Polyethylene glycol octadecyl ether, in about 1.00% by weight of the emulsion;

Poloxamer 188, in about 0.30% by weight of the emulsion;

methylparaben, in about 0.30% by weight of the emulsion;

sodium hydroxide, in about 0.03% by weight of the emulsion;

propylparaben, in about 0.10% by weight of the emulsion;

disodium EDTA, in about 0.10% by weight of the emulsion;

butylated hydroxytoluene, in about 0.03% by weight of the emulsion; and

tretinoin, in about 0.10% by weight of the emulsion.

In certain embodiments, the invention relates to an emulsion, consisting essentially of:

water, in about 69.54% by weight of the emulsion;

oleic acid, in about 8.00% by weight of the emulsion;

diethyl sebacate, in about 4.00% by weight of the emulsion;

glycerin, in about 7.00% by weight of the emulsion;

pentylene glycol, in about 2.50% by weight of the emulsion;

cetostearyl alcohol, in about 3.00% by weight of the emulsion;

emulsifying wax, in about 3.00% by weight of the emulsion;

sodium phosphate monobasic, in about 1.00% by weight of the emulsion;

Polyethylene glycol octadecyl ether, in about 1.00% by weight of the emulsion;

Poloxamer 188, in about 0.30% by weight of the emulsion;

methylparaben, in about 0.30% by weight of the emulsion;

sodium hydroxide, in about 0.03% by weight of the emulsion;

propylparaben, in about 0.10% by weight of the emulsion;

disodium EDTA, in about 0.10% by weight of the emulsion;

butylated hydroxytoluene, in about 0.03% by weight of the emulsion; and

tretinoin, in about 0.10% by weight of the emulsion.

In certain embodiments, the invention relates to an emulsion, consisting of:

water, in about 69.54% by weight of the emulsion;

oleic acid, in about 8.00% by weight of the emulsion;

diethyl sebacate, in about 4.00% by weight of the emulsion;

glycerin, in about 7.00% by weight of the emulsion;

pentylene glycol, in about 2.50% by weight of the emulsion;

cetostearyl alcohol, in about 3.00% by weight of the emulsion;

emulsifying wax, in about 3.00% by weight of the emulsion;

sodium phosphate monobasic, in about 1.00% by weight of the emulsion;

Polyethylene glycol octadecyl ether, in about 1.00% by weight of the emulsion;

Poloxamer 188, in about 0.30% by weight of the emulsion;

methylparaben, in about 0.30% by weight of the emulsion;

sodium hydroxide, in about 0.03% by weight of the emulsion;

propylparaben, in about 0.10% by weight of the emulsion;

disodium EDTA, in about 0.10% by weight of the emulsion;

butylated hydroxytoluene, in about 0.03% by weight of the emulsion; and

tretinoin, in about 0.10% by weight of the emulsion.

In certain embodiments, the invention relates to an emulsion, comprising:

water, in about 64% by weight of the emulsion;

oleic acid, in about 3.3% by weight of the emulsion;

diethyl sebacate, in about 6.7% by weight of the emulsion;

glycerin, in about 7. % by weight of the emulsion;

pentylene glycol, in about 2.5% by weight of the emulsion;

cetostearyl alcohol, in about 3% by weight of the emulsion;

emulsifying wax, in about 3% by weight of the emulsion;

petrolatum, in about 3% by weight of the emulsion;

Ceteth-20, in about 6% by weight of the emulsion;

sodium phosphate monobasic, in about 1% by weight of the emulsion;

Poloxamer 188, in about 0.3% by weight of the emulsion;

methylparaben, in about 0.3% by weight of the emulsion;

sodium hydroxide, in about 0.03% by weight of the emulsion;

propylparaben, in about 0.1% by weight of the emulsion;

disodium EDTA, in about 0.1% by weight of the emulsion;

butylated hydroxytoluene, in about 0.03% by weight of the emulsion; and

tretinoin, in about 0.1% by weight of the emulsion.

In certain embodiments, the invention relates to an emulsion, consisting essentially of:

water, in about 64% by weight of the emulsion;

oleic acid, in about 3.3% by weight of the emulsion;

diethyl sebacate, in about 6.7% by weight of the emulsion;

glycerin, in about 7% by weight of the emulsion;

pentylene glycol, in about 2.5% by weight of the emulsion;

cetostearyl alcohol, in about 3% by weight of the emulsion;

emulsifying wax, in about 3% by weight of the emulsion;

petrolatum, in about 3% by weight of the emulsion;

Ceteth-20, in about 6% by weight of the emulsion;

sodium phosphate monobasic, in about 1% by weight of the emulsion;

Poloxamer 188, in about 0.3% by weight of the emulsion;

methylparaben, in about 0.3% by weight of the emulsion;

sodium hydroxide, in about 0.03% by weight of the emulsion;

propylparaben, in about 0.1% by weight of the emulsion;

disodium EDTA, in about 0.1% by weight of the emulsion;

butylated hydroxytoluene, in about 0.03% by weight of the emulsion; and

tretinoin, in about 0.1% by weight of the emulsion.

In certain embodiments, the invention relates to an emulsion, consisting of:

water, in about 64% by weight of the emulsion;

oleic acid, in about 3.3% by weight of the emulsion;

diethyl sebacate, in about 6.7% by weight of the emulsion;

glycerin, in about 7% by weight of the emulsion;

pentylene glycol, in about 2.5% by weight of the emulsion;

cetostearyl alcohol, in about 3% by weight of the emulsion;

emulsifying wax, in about 3% by weight of the emulsion;

petrolatum, in about 3% by weight of the emulsion;

Ceteth-20, in about 6% by weight of the emulsion;

sodium phosphate monobasic, in about 1% by weight of the emulsion;

Poloxamer 188, in about 0.3% by weight of the emulsion;

methylparaben, in about 0.3% by weight of the emulsion;

sodium hydroxide, in about 0.03% by weight of the emulsion;

propylparaben, in about 0.1% by weight of the emulsion;

disodium EDTA, in about 0.1% by weight of the emulsion;

butylated hydroxytoluene, in about 0.03% by weight of the emulsion; and
tretinoin, in about 0.1% by weight of the emulsion.

In certain embodiments, the invention relates to an emulsion, comprising:

water, in about 63.55% by weight of the emulsion;

oleic acid, in about 3.33% by weight of the emulsion;

diethyl sebacate, in about 6.66% by weight of the emulsion;

glycerin, in about 7.00% by weight of the emulsion;

pentylene glycol, in about 2.50% by weight of the emulsion;

cetostearyl alcohol, in about 3.00% by weight of the emulsion;

emulsifying wax, in about 3.00% by weight of the emulsion;

petrolatum, in about 3.00% by weight of the emulsion;

Ceteth-20, in about 6.00% by weight of the emulsion;

sodium phosphate monobasic, in about 1.00% by weight of the emulsion;

Poloxamer 188, in about 0.30% by weight of the emulsion;

methylparaben, in about 0.30% by weight of the emulsion;

sodium hydroxide, in about 0.03% by weight of the emulsion;

propylparaben, in about 0.10% by weight of the emulsion;

disodium EDTA, in about 0.10% by weight of the emulsion;

butylated hydroxytoluene, in about 0.03% by weight of the emulsion; and

tretinoin, in about 0.10% by weight of the emulsion.

In certain embodiments, the invention relates to an emulsion, consisting essentially of:

water, in about 63.55% by weight of the emulsion;

oleic acid, in about 3.33% by weight of the emulsion;

diethyl sebacate, in about 6.66% by weight of the emulsion;

glycerin, in about 7.00% by weight of the emulsion;

pentylene glycol, in about 2.50% by weight of the emulsion;

cetostearyl alcohol, in about 3.00% by weight of the emulsion;

emulsifying wax, in about 3.00% by weight of the emulsion;

petrolatum, in about 3.00% by weight of the emulsion;

Ceteth-20, in about 6.00% by weight of the emulsion;

sodium phosphate monobasic, in about 1.00% by weight of the emulsion;

Poloxamer 188, in about 0.30% by weight of the emulsion;

methylparaben, in about 0.30% by weight of the emulsion;

sodium hydroxide, in about 0.03% by weight of the emulsion;

propylparaben, in about 0.10% by weight of the emulsion;

disodium EDTA, in about 0.10% by weight of the emulsion;

butylated hydroxytoluene, in about 0.03% by weight of the emulsion; and

tretinoin, in about 0.10% by weight of the emulsion.

In certain embodiments, the invention relates to an emulsion, consisting of:

water, in about 63.55% by weight of the emulsion;

oleic acid, in about 3.33% by weight of the emulsion;

diethyl sebacate, in about 6.66% by weight of the emulsion;

glycerin, in about 7.00% by weight of the emulsion;

pentylene glycol, in about 2.50% by weight of the emulsion;

cetostearyl alcohol, in about 3.00% by weight of the emulsion;

emulsifying wax, in about 3.00% by weight of the emulsion;

petrolatum, in about 3.00% by weight of the emulsion;

Ceteth-20, in about 6.00% by weight of the emulsion;

sodium phosphate monobasic, in about 1.00% by weight of the emulsion;

Poloxamer 188, in about 0.30% by weight of the emulsion;

methylparaben, in about 0.30% by weight of the emulsion;

sodium hydroxide, in about 0.03% by weight of the emulsion;

propylparaben, in about 0.10% by weight of the emulsion;

disodium EDTA, in about 0.10% by weight of the emulsion;

butylated hydroxytoluene, in about 0.03% by weight of the emulsion; and

tretinoin, in about 0.10% by weight of the emulsion.

In certain embodiments, the invention relates to an emulsion, comprising:

water, in about 67% by weight of the emulsion;

diethyl sebacate, in about 8% by weight of the emulsion;

glycerin, in about 7% by weight of the emulsion;

isostearyl alcohol, in about 4% by weight of the emulsion;

pentylene glycol, in about 2.5% by weight of the emulsion;

cetostearyl alcohol, in about 3% by weight of the emulsion;

emulsifying wax, in about 3% by weight of the emulsion;

petrolatum, in about 4% by weight of the emulsion;

sodium phosphate monobasic, in about 1% by weight of the emulsion;

Poloxamer 188, in about 0.3% by weight of the emulsion;

methylparaben, in about 0.3% by weight of the emulsion;

sodium hydroxide, in about 0.03% by weight of the emulsion;

propylparaben, in about 0.1% by weight of the emulsion;

disodium EDTA, in about 0.1% by weight of the emulsion;

butylated hydroxytoluene, in about 0.03% by weight of the emulsion; and

tretinoin, in about 0.1% by weight of the emulsion.

In certain embodiments, the invention relates to an emulsion, consisting essentially of:

water, in about 67% by weight of the emulsion;

diethyl sebacate, in about 8% by weight of the emulsion;

glycerin, in about 7% by weight of the emulsion;

isostearyl alcohol, in about 4% by weight of the emulsion;

pentylene glycol, in about 2.5% by weight of the emulsion;

cetostearyl alcohol, in about 3% by weight of the emulsion;

emulsifying wax, in about 3% by weight of the emulsion;

petrolatum, in about 4% by weight of the emulsion;

sodium phosphate monobasic, in about 1% by weight of the emulsion;

Poloxamer 188, in about 0.3% by weight of the emulsion;

methylparaben, in about 0.3% by weight of the emulsion;

sodium hydroxide, in about 0.03% by weight of the emulsion;

propylparaben, in about 0.1% by weight of the emulsion;

disodium EDTA, in about 0.1% by weight of the emulsion;

butylated hydroxytoluene, in about 0.03% by weight of the emulsion; and

tretinoin, in about 0.1% by weight of the emulsion.

In certain embodiments, the invention relates to an emulsion, consisting of:

water, in about 67% by weight of the emulsion;

diethyl sebacate, in about 8% by weight of the emulsion;

glycerin, in about 7% by weight of the emulsion;

isostearyl alcohol, in about 4% by weight of the emulsion;

pentylene glycol, in about 2.5% by weight of the emulsion;

cetostearyl alcohol, in about 3% by weight of the emulsion;

emulsifying wax, in about 3% by weight of the emulsion;

petrolatum, in about 4% by weight of the emulsion;

sodium phosphate monobasic, in about 1% by weight of the emulsion;

Poloxamer 188, in about 0.3% by weight of the emulsion;

methylparaben, in about 0.3% by weight of the emulsion;

sodium hydroxide, in about 0.03% by weight of the emulsion;

propylparaben, in about 0.1% by weight of the emulsion;

disodium EDTA, in about 0.1% by weight of the emulsion;

butylated hydroxytoluene, in about 0.03% by weight of the emulsion; and

tretinoin, in about 0.1% by weight of the emulsion.

In certain embodiments, the invention relates to an emulsion, comprising:

water, in about 66.54% by weight of the emulsion;

diethyl sebacate, in about 8.00% by weight of the emulsion;

glycerin, in about 7.00% by weight of the emulsion;

isostearyl alcohol, in about 4.00% by weight of the emulsion;

pentylene glycol, in about 2.50% by weight of the emulsion;

cetostearyl alcohol, in about 3.00% by weight of the emulsion;

emulsifying wax, in about 3.00% by weight of the emulsion;

petrolatum, in about 4.00% by weight of the emulsion;

sodium phosphate monobasic, in about 1.00% by weight of the emulsion;

Poloxamer 188, in about 0.30% by weight of the emulsion;

methylparaben, in about 0.30% by weight of the emulsion;

sodium hydroxide, in about 0.03% by weight of the emulsion;

propylparaben, in about 0.10% by weight of the emulsion;

disodium EDTA, in about 0.10% by weight of the emulsion;

butylated hydroxytoluene, in about 0.03% by weight of the emulsion; and

tretinoin, in about 0.10% by weight of the emulsion.

In certain embodiments, the invention relates to an emulsion, consisting essentially of:

water, in about 66.54% by weight of the emulsion;

diethyl sebacate, in about 8.00% by weight of the emulsion;

glycerin, in about 7.00% by weight of the emulsion;

isostearyl alcohol, in about 4.00% by weight of the emulsion;

pentylene glycol, in about 2.50% by weight of the emulsion;

cetostearyl alcohol, in about 3.00% by weight of the emulsion;

emulsifying wax, in about 3.00% by weight of the emulsion;

petrolatum, in about 4.00% by weight of the emulsion;

sodium phosphate monobasic, in about 1.00% by weight of the emulsion;

Poloxamer 188, in about 0.30% by weight of the emulsion;

methylparaben, in about 0.30% by weight of the emulsion;

sodium hydroxide, in about 0.03% by weight of the emulsion;

propylparaben, in about 0.10% by weight of the emulsion;

disodium EDTA, in about 0.10% by weight of the emulsion;

butylated hydroxytoluene, in about 0.03% by weight of the emulsion; and

tretinoin, in about 0.10% by weight of the emulsion.

In certain embodiments, the invention relates to an emulsion, consisting of:

water, in about 66.54% by weight of the emulsion;

diethyl sebacate, in about 8.00% by weight of the emulsion;

glycerin, in about 7.00% by weight of the emulsion;

isostearyl alcohol, in about 4.00% by weight of the emulsion;

pentylene glycol, in about 2.50% by weight of the emulsion;

cetostearyl alcohol, in about 3.00% by weight of the emulsion;

emulsifying wax, in about 3.00% by weight of the emulsion;

petrolatum, in about 4.00% by weight of the emulsion;

sodium phosphate monobasic, in about 1.00% by weight of the emulsion;

Poloxamer 188, in about 0.30% by weight of the emulsion;

methylparaben, in about 0.30% by weight of the emulsion;

sodium hydroxide, in about 0.03% by weight of the emulsion;

propylparaben, in about 0.10% by weight of the emulsion;

disodium EDTA, in about 0.10% by weight of the emulsion;

butylated hydroxytoluene, in about 0.03% by weight of the emulsion; and

tretinoin, in about 0.10% by weight of the emulsion.

Exemplary Components of the Emulsion

As outlined above, in certain embodiments, the invention relates to an emulsion comprising water, an emulsifier or surfactant, a moisturizer or emollient, an antioxidant or preservative, and an active agent. The components described below may be present in any one of the aforementioned emulsions.

In certain embodiments, the invention relates to any one of the aforementioned emulsions, wherein the emulsifier or surfactant is selected from the group consisting of: polysorbate 20, polysorbate 40, polysorbate 60, polysorbate 80, steareth-10, sodium dodecyl sulfate, lauryl dimethyl amine oxide, cetyltrimethylammonium bromide, polyethoxylated alcohols, polyoxyethylene sorbitan, octoxynol, N,N-dimethyldodecylamine-N-oxide, hexadecyltrimethylammonium bromide, polyoxyl 10 lauryl ether, sodium deoxycholate, sodium cholate, polyoxyl castor oil, nonylphenol ethoxylate, cyclodextrins, lecithin, dimethicone copolyol, lauramide DEA, cocamide DEA, cocamide MEA, oleyl betaine, cocamidopropyl betaine, cocamidopropyl phosphatidyl PG-dimonium chloride, dicetyl phosphate, ceteareth-10 phosphate, methylbenzethonium chloride, behentrimonium methosulfate-cetearyl alcohol, non-ionic emulsifiers like emulsifying wax, polyoxyethylene oleyl ether, PEG-40 stearate, ceteareth-12, ceteareth-20, ceteareth-30, ceteareth alcohol, Ceteth-20, oleic acid, oleyl alcohol, glyceryl stearate, PEG-100 stearate, glyceryl stearate and PEG-100 stearate, steareth-2, steareth-20, stearic acid, cholesterol, ceramide 2, ceramide 3, stearamidopropyl dimethylamine, behentrimonium methosulfate, cetostearyl alcohol, dicetyl phosphate, ceteth-10 phosphate, polyethylene glycol octadecyl ether, Poloxamer 188, and combinations/mixtures thereof.

In certain embodiments, the invention relates to any one of the aforementioned emulsions, wherein the emulsifier or surfactant is selected from the group consisting of: emulsifying wax, cetostearyl alcohol, Ceteth-20, oleic acid, dicetyl phosphate, ceteth-10 phosphate, polyethylene glycol octadecyl ether, Poloxamer 188, and combinations/mixtures thereof.

In certain embodiments, the invention relates to any one of the aforementioned emulsions, wherein the emulsifier or surfactant is present in an amount from about 1% to about 25% by weight of the emulsion. In certain embodiments, the emulsifier or surfactant is present in an amount from about 2% to about 20% by weight of the emulsion. In certain embodiments, the emulsifier or surfactant is present in an amount of about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight of the emulsion.

In certain embodiments, the invention relates to any one of the aforementioned emulsions, wherein the emulsifier or surfactant comprises oleic acid. In certain embodiments, the oleic acid is present in an amount from about 1.5% to about 14% by weight of the emulsion. In certain embodiments, the oleic acid is present in an amount from about 2% to about 12% by weight of the emulsion. In certain embodiments, the oleic acid is present in about 2%, about 3%, about 4%, about 5%, about 6%, about 7%, about 8%, about 9%, about 10%, about 11%, or about 12% by weight of the emulsion.

In certain embodiments, the invention relates to any one of the aforementioned emulsions, wherein the emulsifier or surfactant comprises oleic acid. In certain embodiments, the oleic acid is present in an amount from about 1.50% to about 14.00% by weight of the emulsion. In certain embodiments, the oleic acid is present in an amount from about 2.00% to about 12.00% by weight of the emulsion. In certain embodiments, the oleic acid is present in about 2.00%, about 3.00%, about 4.00%, about 5.00%, about 6.00%, about 7.00%, about 8.00%, about 9.00%, about 10.00%, about 11.00%, or about 12.00% by weight of the emulsion.

In certain embodiments, the invention relates to any one of the aforementioned emulsions, wherein the emulsifier or surfactant comprises dicetyl phosphate. In certain embodiments, the dicetyl phosphate is present in an amount from about 1% to about 3% by weight of the emulsion. In certain embodiments, the dicetyl phosphate is present in an amount from about 1.5% to about 2.5% by weight of the emulsion. In certain embodiments, the dicetyl phosphate is present in about 1.5%, about 1.6%, about 1.7%, about 1.8%, about 1.9%, about 2.0%, about 2.1%, about 2.2%, about 2.3%, about 2.4%, or about 2.5% by weight of the emulsion.

In certain embodiments, the invention relates to any one of the aforementioned emulsions, wherein the emulsifier or surfactant comprises dicetyl phosphate. In certain embodiments, the dicetyl phosphate is present in an amount from about 1.00% to about 3.00% by weight of the emulsion. In certain embodiments, the dicetyl phosphate is present in an amount from about 1.50% to about 2.50% by weight of the emulsion. In certain embodiments, the dicetyl phosphate is present in about 1.50%, about 1.60%, about 1.70%, about 1.80%, about 1.90%, about 2.00%, about 2.10%, about 2.20%, about 2.30%, about 2.40%, or about 2.50% by weight of the emulsion.

In certain embodiments, the invention relates to any one of the aforementioned emulsions, wherein the emulsifier or surfactant comprises ceteth-10 phosphate. In certain embodiments, the ceteth-10 phosphate is present in an amount from 0.5% to about 1.5% by weight of the emulsion. In certain embodiments, the ceteth-10 phosphate is present in an amount from about 0.6% to about 1.4% by weight of the emulsion. In certain embodiments, the ceteth-10 phosphate is present in about 0.6%, about 0.7%, about 0.8%, about 0.9%, about 1.0%, about 1.1%, about 1.2%, about 1.3%, or about 1.4% by weight of the emulsion.

In certain embodiments, the invention relates to any one of the aforementioned emulsions, wherein the emulsifier or surfactant comprises ceteth-10 phosphate. In certain embodiments, the ceteth-10 phosphate is present in an amount from 0.50% to about 1.50% by weight of the emulsion. In certain embodiments, the ceteth-10 phosphate is present in an amount from about 0.60% to about 1.40% by weight of the emulsion. In certain embodiments, the ceteth-10 phosphate is present in about 0.60%, about 0.70%, about 0.80%, about 0.90%, about 1.00%, about 1.10%, about 1.20%, about 1.30%, or about 1.40% by weight of the emulsion.

In certain embodiments, the invention relates to any one of the aforementioned emulsions, wherein the emulsifier or surfactant comprises cetostearyl alcohol. In certain embodiments, the cetostearyl alcohol is present in an amount from about 0.5% to about 5% by weight of the emulsion. In certain embodiments, the cetostearyl alcohol is present in an amount from about 1% to about 4% by weight of the emulsion. In certain embodiments, the cetostearyl alcohol is present in about 1%, about 2%, about 3%, or about 4% by weight of the emulsion.

In certain embodiments, the invention relates to any one of the aforementioned emulsions, wherein the emulsifier or surfactant comprises cetostearyl alcohol. In certain embodiments, the cetostearyl alcohol is present in an amount from about 0.50% to about 5.00% by weight of the emulsion. In certain embodiments, the cetostearyl alcohol is present in an amount from about 1.00% to about 4.00% by weight of the emulsion. In certain embodiments, the cetostearyl alcohol is present in about 1.00%, about 2.00%, about 3.00%, or about 4.00% by weight of the emulsion.

In certain embodiments, the invention relates to any one of the aforementioned emulsions, wherein the emulsifier or surfactant comprises emulsifying wax. In certain embodiments, the emulsifying wax is present in an amount from about 0.5% to about 5% by weight of the emulsion. In certain embodiments, the emulsifying wax is present in an amount from about 1% to about 4% by weight of the emulsion. In certain embodiments, the emulsifying wax is present in about 1%, about 2%, about 3%, or about 4% by weight of the emulsion.

In certain embodiments, the invention relates to any one of the aforementioned emulsions, wherein the emulsifier or surfactant comprises emulsifying wax. In certain embodiments, the emulsifying wax is present in an amount from about 0.50% to about 5.00% by weight of the emulsion. In certain embodiments, the emulsifying wax is present in an amount from about 1.00% to about 4.00% by weight of the emulsion. In certain embodiments, the emulsifying wax is present in about 1.00%, about 2.00%, about 3.00%, or about 4.00% by weight of the emulsion.

In certain embodiments, the invention relates to any one of the aforementioned emulsions, wherein the emulsifier or surfactant comprises Ceteth-20. In certain embodiments, the Ceteth-20 is present in an amount from about 3% to about 9% by weight of the emulsion. In certain embodiments, the Ceteth-20 is present in an amount from about 4% to about 8% by weight of the emulsion. In certain embodiments, the Ceteth-20 is present in about 4%, about 5%, about 6%, about 7%, or about 8% by weight of the emulsion.

In certain embodiments, the invention relates to any one of the aforementioned emulsions, wherein the emulsifier or surfactant comprises Ceteth-20. In certain embodiments, the Ceteth-20 is present in an amount from about 3.00% to about 9.00% by weight of the emulsion. In certain embodiments, the Ceteth-20 is present in an amount from about 4.00% to about 8.00% by weight of the emulsion. In certain embodiments, the Ceteth-20 is present in about 4.00%, about 5.00%, about 6.00%, about 7.00%, or about 8.00% by weight of the emulsion.

In certain embodiments, the invention relates to any one of the aforementioned emulsions, wherein the emulsifier or surfactant comprises polyethylene glycol octadecyl ether. In certain embodiments, the polyethylene glycol octadecyl ether is present in an amount from about 0.2% to about 3% by weight of the emulsion. In certain embodiments, the polyethylene glycol octadecyl ether is present in an amount from about 0.3% to about 2.5% by weight of the emulsion. In certain embodiments, the polyethylene glycol octadecyl ether is present in about 0.3%, about 0.4%, about 0.5%, about 0.6%, about 0.7%, about 0.8%, about 0.9%, about 1%, about 1.25%, about 1.5%, about 1.75%, about 2%, about 2.25%, or about 2.5% by weight of the emulsion.

In certain embodiments, the invention relates to any one of the aforementioned emulsions, wherein the emulsifier or surfactant comprises polyethylene glycol octadecyl ether. In certain embodiments, the polyethylene glycol octadecyl ether is present in an amount from about 0.20% to about 3.00% by weight of the emulsion. In certain embodiments, the polyethylene glycol octadecyl ether is present in an amount from about 0.30% to about 2.50% by weight of the emulsion. In certain embodiments, the Polyethylene glycol octadecyl ether is present in about 0.30%, about 0.40%, about 0.50%, about 0.60%, about 0.70%, about 0.80%, about 0.90%, about 1.00%, about 1.25%, about 1.50%, about 1.75%, about 2.00%, about 2.25%, or about 2.50% by weight of the emulsion.

In certain embodiments, the invention relates to any one of the aforementioned emulsions, wherein the emulsifier or surfactant comprises Poloxamer 188. In certain embodiments, the Poloxamer 188 is present in an amount from about 0.01% to about 2% by weight of the emulsion. In certain embodiments, the Poloxamer 188 is present in an amount from about 0.15% to about 0.45% by weight of the emulsion. In certain embodiments, the Poloxamer 188 is present in about 0.15%, about 0.2%, about 0.25%, about 0.3%, about 0.35%, about 0.4%, or about 0.45% by weight of the emulsion.

In certain embodiments, the invention relates to any one of the aforementioned emulsions, wherein the emulsifier or surfactant comprises Poloxamer 188. In certain embodiments, the Poloxamer 188 is present in an amount from about 0.01% to about 2.00% by weight of the emulsion. In certain embodiments, the Poloxamer 188 is present in an amount from about 0.15% to about 0.45% by weight of the emulsion. In certain embodiments, the Poloxamer 188 is present in about 0.15%, about 0.20%, about 0.25%, about 0.30%, about 0.35%, about 0.40%, or about 0.45% by weight of the emulsion.

In certain embodiments, the invention relates to any one of the aforementioned emulsions, wherein the moisturizer or emollient is selected from the group consisting of petrolatum, lactic acid, glycerol, butylene glycol, sodium PCA, sodium hyaluronate, Carbowax 200, Carbowax 400, Carbowax 800, cetyl palmitate, PPG-15 stearyl ether, lanolin alcohol, lanolin, lanolin derivatives, cholesterol, isostearyl neopentanoate, octyl stearate, mineral oil, isocetyl stearate, myristyl myristate, octyl dodecanol, 2-ethylhexyl palmitate, dimethicone, phenyl trimethicone, cyclomethicone, C₁₂-C₁₅ alkyl benzoates, dimethiconol, propylene glycol, Theobroma grandiflorum seed butter, ceramide 2, hydroxypropyl bispalmitamide MEA, hydroxypropyl bislauramide MEA, hydroxypropyl bisisostearamide MEA, 1,3-bis(N-2-(hydroxyethyl)stearoylamino)-2-hydroxy propane, bis-hydroxyethyl tocopherylsuccinoylamido hydroxypropane, urea, aloe, allantoin, glycyrrhetinic acid, safflower oil, oleyl alcohol, stearic acid, dicaprylate/dicaprate, diethyl sebacate, isostearyl alcohol, pentylene glycol, 1,3-bis(N-2-(hydroxyethyl)palmitoylamino)-2-hydroxypropane, and combinations/mixtures thereof.

In certain embodiments, the invention relates to any one of the aforementioned emulsions, wherein the moisturizer or emollient is selected from the group consisting of petrolatum, glycerol (glycerin), sodium hyaluronate, 2-ethylhexyl palmitate, dimethicone, C₁₂-C₁₅ alkyl benzoates, propylene glycol, Theobroma grandiflorum seed butter, diethyl sebacate, isostearyl alcohol, pentylene glycol, 1,3-bis(N-2-(hydroxyethyl)palmitoylamino)-2-hydroxypropane, and combinations/mixtures thereof.

In certain embodiments, the invention relates to any one of the aforementioned emulsions, wherein the moisturizer or emollient is present in an amount from about 4% to about 30% by weight of the emulsion. In certain embodiments, the moisturizer or emollient is present in an amount from about 6% to about 25% by weight of the emulsion. In certain embodiments, the moisturizer or emollient is present in an amount of about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% by weight of the emulsion.

In certain embodiments, the invention relates to any one of the aforementioned emulsions, wherein the moisturizer or emollient comprises sodium hyaluronate. In certain embodiments, the sodium hyaluronate is present in an amount from about 0.05% to about 1.5% by weight of the emulsion. In certain embodiments, the sodium hyaluronate is present in an amount from about 0.07% to about 0.13% by weight of the emulsion. In certain embodiments, the sodium hyaluronate is present in about 0.07%, about 0.08%, about 0.09%, about 0.10%, about 0.11%, about 0.12%, or about 0.13% by weight of the emulsion.

In certain embodiments, the invention relates to any one of the aforementioned emulsions, wherein the moisturizer or emollient comprises diethyl sebacate. In certain embodiments, the diethyl sebacate is present in an amount from about 2% to about 12% by weight of the emulsion. In certain embodiments, the diethyl sebacate is present in an amount from about 3% to about 11% by weight of the emulsion. In certain embodiments, the diethyl sebacate is present in about 3%, about 4%, about 5%, about 6%, about 7%, about 8%, about 9%, about 10% or about 11% by weight of the emulsion.

In certain embodiments, the invention relates to any one of the aforementioned emulsions, wherein the moisturizer or emollient comprises diethyl sebacate. In certain embodiments, the diethyl sebacate is present in an amount from about 2.00% to about 12.00% by weight of the emulsion. In certain embodiments, the diethyl sebacate is present in an amount from about 3.00% to about 11.00% by weight of the emulsion. In certain embodiments, the diethyl sebacate is present in about 3.00%, about 4.00%, about 5.00%, about 6.00%, about 7.00%, about 8.00%, about 9.00%, about 10.00% or about 11.00% by weight of the emulsion.

In certain embodiments, the invention relates to any one of the aforementioned emulsions, wherein the moisturizer or emollient comprises glycerin. In certain embodiments, the glycerin is present in an amount from about 2.5% to about 10% by weight of the emulsion. In certain embodiments, the glycerin is present in an amount from about 3% to about 9% by weight of the emulsion. In certain embodiments, the glycerin is present in about 3%, about 4%, about 5%, about 6%, about 7%, about 8%, or about 9% by weight of the emulsion.

In certain embodiments, the invention relates to any one of the aforementioned emulsions, wherein the moisturizer or emollient comprises glycerin. In certain embodiments, the glycerin is present in an amount from about 2.50% to about 10.00% by weight of the emulsion. In certain embodiments, the glycerin is present in an amount from about 3.00% to about 9.00% by weight of the emulsion. In certain embodiments, the glycerin is present in about 3.00%, about 4.00%, about 5.00%, about 6.00%, about 7.00%, about 8.00%, or about 9.00% by weight of the emulsion.

In certain embodiments, the invention relates to any one of the aforementioned emulsions, wherein the moisturizer or emollient comprises ethyl hexyl palmitate. In certain embodiments, the ethyl hexyl palmitate is present in an amount from about 3% to about 9% by weight of the emulsion. In certain embodiments, the ethyl hexyl palmitate is present in an amount from about 4% to about 8% by weight of the emulsion. In certain embodiments, the ethyl hexyl palmitate is present in about 4%, about 5%, about 6%, about 7%, or about 8% by weight of the emulsion.

In certain embodiments, the invention relates to any one of the aforementioned emulsions, wherein the moisturizer or emollient comprises ethyl hexyl palmitate. In certain embodiments, the ethyl hexyl palmitate is present in an amount from about 3.00% to about 9.00% by weight of the emulsion. In certain embodiments, the ethyl hexyl palmitate is present in an amount from about 4.00% to about 8.00% by weight of the emulsion. In certain embodiments, the ethyl hexyl palmitate is present in about 4.00%, about 5.00%, about 6.00%, about 7.00%, or about 8.00% by weight of the emulsion.

In certain embodiments, the invention relates to any one of the aforementioned emulsions, wherein the moisturizer or emollient comprises isostearyl alcohol. In certain embodiments, the isostearyl alcohol is present in an amount from about 2% to about 6% by weight of the emulsion. In certain embodiments, the isostearyl alcohol is present in an amount from about 2.5% to about 5.5% by weight of the emulsion. In certain embodiments, the isostearyl alcohol is present in about 2.5%, about 3%, about 3.5%, about 4%, about 4.5%, about 5%, or about 5.5% by weight of the emulsion.

In certain embodiments, the invention relates to any one of the aforementioned emulsions, wherein the moisturizer or emollient comprises isostearyl alcohol. In certain embodiments, the isostearyl alcohol is present in an amount from about 2.00% to about 6.00% by weight of the emulsion. In certain embodiments, the isostearyl alcohol is present in an amount from about 2.50% to about 5.50% by weight of the emulsion. In certain embodiments, the isostearyl alcohol is present in about 2.50%, about 3.00%, about 3.50%, about 4.00%, about 4.50%, about 5.00%, or about 5.50% by weight of the emulsion.

In certain embodiments, the invention relates to any one of the aforementioned emulsions, wherein the moisturizer or emollient comprises pentylene glycol. In certain embodiments, the pentylene glycol is present in an amount from about 1% to about 4% by weight of the emulsion. In certain embodiments, the pentylene glycol is present in an amount from about 1.5% to about 3.5% by weight of the emulsion. In certain embodiments, the pentylene glycol is present in about 1.5%, about 2%, about 2.5%, about 3%, or about 3.5% by weight of the emulsion.

In certain embodiments, the invention relates to any one of the aforementioned emulsions, wherein the moisturizer or emollient comprises pentylene glycol. In certain embodiments, the pentylene glycol is present in an amount from about 1.00% to about 4.00% by weight of the emulsion. In certain embodiments, the pentylene glycol is present in an amount from about 1.50% to about 3.50% by weight of the emulsion. In certain embodiments, the pentylene glycol is present in about 1.50%, about 2.00%, about 2.50%, about 3.00%, or about 3.50% by weight of the emulsion.

In certain embodiments, the invention relates to any one of the aforementioned emulsions, wherein the moisturizer or emollient comprises propylene glycol. In certain embodiments, the propylene glycol is present in an amount from about 1% to about 4% by weight of the emulsion. In certain embodiments, the propylene glycol is present in an amount from about 1.5% to about 3.5% by weight of the emulsion. In certain embodiments, the propylene glycol is present in about 1.5%, about 2%, about 2.5%, about 3%, or about 3.5% by weight of the emulsion.

In certain embodiments, the invention relates to any one of the aforementioned emulsions, wherein the moisturizer or emollient comprises propylene glycol. In certain embodiments, the propylene glycol is present in an amount from about 1.00% to about 4.00% by weight of the emulsion. In certain embodiments, the propylene glycol is present in an amount from about 1.50% to about 3.50% by weight of the emulsion. In certain embodiments, the propylene glycol is present in about 1.50%, about 2.00%, about 2.50%, about 3.00%, or about 3.50% by weight of the emulsion.

In certain embodiments, the invention relates to any one of the aforementioned emulsions, wherein the moisturizer or emollient comprises Theobroma grandiflorum seed butter. In certain embodiments, the Theobroma grandiflorum seed butter is present in an amount from about 1% to about 3% by weight of the emulsion. In certain embodiments, the Theobroma grandiflorum seed butter is present in an amount from about 1.5% to about 2.5% by weight of the emulsion. In certain embodiments, the Theobroma grandiflorum seed butter is present in about 1.5%, about 2%, or about 2.5% by weight of the emulsion.

In certain embodiments, the invention relates to any one of the aforementioned emulsions, wherein the moisturizer or emollient comprises Theobroma grandiflorum seed butter. In certain embodiments, the Theobroma grandiflorum seed butter is present in an amount from about 1.00% to about 3.00% by weight of the emulsion. In certain embodiments, the Theobroma grandiflorum seed butter is present in an amount from about 1.50% to about 2.50% by weight of the emulsion. In certain embodiments, the Theobroma grandiflorum seed butter is present in about 1.50%, about 2.00%, or about 2.50% by weight of the emulsion.

In certain embodiments, the invention relates to any one of the aforementioned emulsions, wherein the moisturizer or emollient comprises dimethicone. In certain embodiments, the dimethicone is present in an amount from about 0.2% to about 2% by weight of the emulsion. In certain embodiments, the dimethicone is present in an amount from about 0.3% to about 1.5% by weight of the emulsion. In certain embodiments, the dimethicone is present in about 0.3%, about 0.4%, about 0.5%, about 0.6%, about 0.7%, about 0.8%, about 0.9%, about 1%, about 1.1%, about 1.2%, about 1.3%, about 1.4%, or about 1.5% by weight of the emulsion.

In certain embodiments, the invention relates to any one of the aforementioned emulsions, wherein the moisturizer or emollient comprises dimethicone. In certain embodiments, the dimethicone is present in an amount from about 0.20% to about 2.00% by weight of the emulsion. In certain embodiments, the dimethicone is present in an amount from about 0.30% to about 1.50% by weight of the emulsion. In certain embodiments, the dimethicone is present in about 0.30%, about 0.40%, about 0.50%, about 0.60%, about 0.70%, about 0.80%, about 0.90%, about 1.00%, about 1.10%, about 1.20%, about 1.30%, about 1.40%, or about 1.50% by weight of the emulsion.

In certain embodiments, the invention relates to any one of the aforementioned emulsions, wherein the moisturizer or emollient comprises petrolatum. In certain embodiments, the petrolatum is present in an amount from about 0.5% to about 6% by weight of the emulsion. In certain embodiments, the petrolatum is present in an amount from about 0.8% to about 5% by weight of the emulsion. In certain embodiments, the petrolatum is present in about 0.8%, about 1%, about 1.5%, about 2%, about 2.5%, about 3%, about 3.5%, about 4%, about 4.5%, or about 5% by weight of the emulsion. In certain embodiments, the petrolatum is white petrolatum.

In certain embodiments, the invention relates to any one of the aforementioned emulsions, wherein the moisturizer or emollient comprises petrolatum. In certain embodiments, the petrolatum is present in an amount from about 0.50% to about 6.00% by weight of the emulsion. In certain embodiments, the petrolatum is present in an amount from about 0.80% to about 5.00% by weight of the emulsion. In certain embodiments, the petrolatum is present in about 0.80%, about 1.00%, about 1.50%, about 2.00%, about 2.50%, about 3.00%, about 3.50%, about 4.00%, about 4.50%, or about 5.00% by weight of the emulsion. In certain embodiments, the petrolatum is white petrolatum.

In certain embodiments, the invention relates to any one of the aforementioned emulsions, wherein the moisturizer or emollient comprises 1,3-bis(N-2-(hydroxyethyl)palmitoylamino)-2-hydroxypropane. In certain embodiments, the 1,3-bis(N-2-(hydroxyethyl)palmitoylamino)-2-hydroxypropane is present in an amount from about 0.2% to about 0.8% by weight of the emulsion. In certain embodiments, the 1,3-bis(N-2-(hydroxyethyl)palmitoylamino)-2-hydroxypropane is present in an amount from about 0.3% to about 0.7% by weight of the emulsion. In certain embodiments, the 1,3-bis(N-2-(hydroxyethyl)palmitoylamino)-2-hydroxypropane is present in about 0.3%, about 0.4%, about 0.5%, about 0.6%, about 0.7%, or about 0.8% by weight of the emulsion.

In certain embodiments, the invention relates to any one of the aforementioned emulsions, wherein the moisturizer or emollient comprises 1,3-bis(N-2-(hydroxyethyl)palmitoylamino)-2-hydroxypropane. In certain embodiments, the 1,3-bis(N-2-(hydroxyethyl)palmitoylamino)-2-hydroxypropane is present in an amount from about 0.20% to about 0.80% by weight of the emulsion. In certain embodiments, the 1,3-bis(N-2-(hydroxyethyl)palmitoylamino)-2-hydroxypropane is present in an amount from about 0.30% to about 0.70% by weight of the emulsion. In certain embodiments, the 1,3-bis(N-2-(hydroxyethyl)palmitoylamino)-2-hydroxypropane is present in about 0.30%, about 0.40%, about 0.50%, about 0.60%, about 0.70%, or about 0.80% by weight of the emulsion.

In certain embodiments, the invention relates to any one of the aforementioned emulsions, wherein the moisturizer or emollient comprises C₁₂-C₁₅ alkyl benzoates. In certain embodiments, the C₁₂-C₁₅ alkyl benzoates is present in an amount from about 0.1% to about 0.3% by weight of the emulsion. In certain embodiments, the C₁₂-C₁₅ alkyl benzoates is present in an amount from about 0.15% to about 0.25% by weight of the emulsion. In certain embodiments, the C₁₂-C₁₅ alkyl benzoates is present in about 0.15%, about 0.2%, or about 0.25% by weight of the emulsion.

In certain embodiments, the invention relates to any one of the aforementioned emulsions, wherein the moisturizer or emollient comprises C₁₂-C₁₅ alkyl benzoates. In certain embodiments, the C₁₂-C₁₅ alkyl benzoates is present in an amount from about 0.10% to about 0.30% by weight of the emulsion. In certain embodiments, the C₁₂-C₁₅ alkyl benzoates is present in an amount from about 0.15% to about 0.25% by weight of the emulsion. In certain embodiments, the C₁₂-C₁₅ alkyl benzoates is present in about 0.15%, about 0.20%, or about 0.25% by weight of the emulsion.

In certain embodiments, the invention relates to any one of the aforementioned emulsions, wherein the antioxidant or preservative is selected from the group consisting of imidazolidinyl urea, diazolidinyl urea, phenoxyethanol, sodium methyl paraben, methylparaben, ethylparaben, propylparaben, potassium sorbate, sodium benzoate, sorbic acid, benzoic acid, formaldehyde, citric acid, sodium citrate, chlorine dioxide, benzalkonium chloride, benzethonium chloride, cetrimide, dequalinium chloride, cetylpyridinium chloride, phenylmercuric nitrate, phenylmercuric acetate, thimerosal, piroctone olamine, Vitis vinifera seed oil, chlorobutanol, dichlorobenzyl alcohol, phenylethyl alcohol, benzyl alcohol, ascorbic acid, sodium bisulfite, butylated hydroxytoluene, butylated hydroxyanisole, α-tocopherol, tocopheryl acetate, sodium ascorbate/ascorbic acid, ascorbyl palmitate, propyl gallate, disodium EDTA, citric acid, and sodium citrate, and combinations/mixtures thereof.

In certain embodiments, the invention relates to any one of the aforementioned emulsions, wherein the antioxidant or preservative comprises sodium citrate, tocopheryl acetate, methylparaben, propylparaben, disodium EDTA, butylated hydroxytoluene, or citric acid.

In certain embodiments, the invention relates to any one of the aforementioned emulsions, wherein the antioxidant or preservative is present in an amount from about 0.25% to about 2% by weight of the emulsion. In certain embodiments, the antioxidant or preservative is present in an amount from about 0.3% to about 1.9% by weight of the emulsion. In certain embodiments, the antioxidant or preservative is present in an amount of about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, or about 1.9% by weight of the emulsion.

In certain embodiments, the invention relates to any one of the aforementioned emulsions, wherein the antioxidant or preservative is present in an amount from about 0.25% to about 2.00% by weight of the emulsion. In certain embodiments, the antioxidant or preservative is present in an amount from about 0.30% to about 1.90% by weight of the emulsion. In certain embodiments, the antioxidant or preservative is present in an amount of about 0.30%, about 0.40%, about 0.50%, about 0.60%, about 0.70%, about 0.80%, about 0.90%, about 1.00%, about 1.10%, about 1.20%, about 1.30%, about 1.40%, about 1.50%, about 1.60%, about 1.70%, about 1.80%, or about 1.90% by weight of the emulsion.

In certain embodiments, the invention relates to any one of the aforementioned emulsions, wherein the antioxidant or preservative comprises sodium citrate. In certain embodiments, the sodium citrate is present in an amount from about 0.3% to about 0.9% by weight of the emulsion. In certain embodiments, the sodium citrate is present in an amount from about 0.4% to about 0.8% by weight of the emulsion. In certain embodiments, the sodium citrate is present in about 0.4%, about 0.5%, about 0.6%, about 0.7%, or about 0.8% by weight of the emulsion.

In certain embodiments, the invention relates to any one of the aforementioned emulsions, wherein the antioxidant or preservative comprises sodium citrate. In certain embodiments, the sodium citrate is present in an amount from about 0.30% to about 0.90% by weight of the emulsion. In certain embodiments, the sodium citrate is present in an amount from about 0.40% to about 0.80% by weight of the emulsion. In certain embodiments, the sodium citrate is present in about 0.40%, about 0.50%, about 0.60%, about 0.70%, or about 0.80% by weight of the emulsion.

In certain embodiments, the invention relates to any one of the aforementioned emulsions, wherein the antioxidant or preservative comprises tocopheryl acetate. In certain embodiments, the tocopheryl acetate is present in an amount from about 0.2% to about 0.8% by weight of the emulsion. In certain embodiments, the tocopheryl acetate is present in an amount from about 0.3% to about 0.7% by weight of the emulsion. In certain embodiments, the tocopheryl acetate is present in about 0.3%, about 0.4%, about 0.5%, about 0.6%, or about 0.7% by weight of the emulsion.

In certain embodiments, the invention relates to any one of the aforementioned emulsions, wherein the antioxidant or preservative comprises tocopheryl acetate. In certain embodiments, the tocopheryl acetate is present in an amount from about 0.20% to about 0.80% by weight of the emulsion. In certain embodiments, the tocopheryl acetate is present in an amount from about 0.30% to about 0.70% by weight of the emulsion. In certain embodiments, the tocopheryl acetate is present in about 0.30%, about 0.40%, about 0.50%, about 0.60%, or about 0.70% by weight of the emulsion.

In certain embodiments, the invention relates to any one of the aforementioned emulsions, wherein the antioxidant or preservative comprises methylparaben. In certain embodiments, the methylparaben is present in an amount from about 0.01% to about 0.5% by weight of the emulsion. In certain embodiments, the methylparaben is present in an amount from about 0.2% to about 0.4% by weight of the emulsion. In certain embodiments, the methylparaben is present in about 0.2%, about 0.3%, or about 0.4% by weight of the emulsion.

In certain embodiments, the invention relates to any one of the aforementioned emulsions, wherein the antioxidant or preservative comprises methylparaben. In certain embodiments, the methylparaben is present in an amount from about 0.0d1% to about 0.50% by weight of the emulsion. In certain embodiments, the methylparaben is present in an amount from about 0.20% to about 0.40% by weight of the emulsion. In certain embodiments, the methylparaben is present in about 0.20%, about 0.30%, or about 0.40% by weight of the emulsion.

In certain embodiments, the invention relates to any one of the aforementioned emulsions, wherein the antioxidant or preservative comprises propylparaben. In certain embodiments, the propylparaben is present in an amount from about 0.05% to about 0.2% by weight of the emulsion. In certain embodiments, the propylparaben is present in an amount from about 0.07% to about 0.13% by weight of the emulsion. In certain embodiments, the propylparaben is present in about 0.07%, about 0.08%, about 0.09%, about 0.10%, about 0.11%, about 0.12%, or about 0.13% by weight of the emulsion.

In certain embodiments, the invention relates to any one of the aforementioned emulsions, wherein the antioxidant or preservative comprises propylparaben. In certain embodiments, the propylparaben is present in an amount from about 0.05% to about 0.20% by weight of the emulsion. In certain embodiments, the propylparaben is present in an amount from about 0.07% to about 0.13% by weight of the emulsion. In certain embodiments, the propylparaben is present in about 0.07%, about 0.08%, about 0.09%, about 0.10%, about 0.11%, about 0.12%, or about 0.13% by weight of the emulsion.

In certain embodiments, the invention relates to any one of the aforementioned emulsions, wherein the antioxidant or preservative comprises disodium EDTA. In certain embodiments, the disodium EDTA is present in an amount from about 0.05% to about 0.2% by weight of the emulsion. In certain embodiments, the disodium EDTA is present in an amount from about 0.07% to about 0.13% by weight of the emulsion. In certain embodiments, the disodium EDTA is present in about 0.07%, about 0.08%, about 0.09%, about 0.10%, about 0.11%, about 0.12%, or about 0.13% by weight of the emulsion.

In certain embodiments, the invention relates to any one of the aforementioned emulsions, wherein the antioxidant or preservative comprises disodium EDTA. In certain embodiments, the disodium EDTA is present in an amount from about 0.05% to about 0.20% by weight of the emulsion. In certain embodiments, the disodium EDTA is present in an amount from about 0.07% to about 0.13% by weight of the emulsion. In certain embodiments, the disodium EDTA is present in about 0.07%, about 0.08%, about 0.09%, about 0.10%, about 0.11%, about 0.12%, or about 0.13% by weight of the emulsion.

In certain embodiments, the invention relates to any one of the aforementioned emulsions, wherein the antioxidant or preservative comprises butylated hydroxytoluene. In certain embodiments, the butylated hydroxytoluene is present in an amount from about 0.01% to about 0.08% by weight of the emulsion. In certain embodiments, the butylated hydroxytoluene is present in an amount from about 0.02% to about 0.07% by weight of the emulsion. In certain embodiments, the butylated hydroxytoluene is present in about 0.02%, about 0.03%, about 0.04%, about 0.05%, about 0.06%, or about 0.07% by weight of the emulsion.

In certain embodiments, the invention relates to any one of the aforementioned emulsions, wherein the antioxidant or preservative comprises citric acid. In certain embodiments, the citric acid is present in an amount from about 0.02% to about 0.08% by weight of the emulsion. In certain embodiments, the citric acid is present in an amount from about 0.03% to about 0.07% by weight of the emulsion. In certain embodiments, the citric acid is present in about 0.03%, about 0.04%, about 0.05%, about 0.06%, or about 0.07% by weight of the emulsion.

In certain embodiments, the invention relates to any one of the aforementioned emulsions, wherein water is present in an amount about 24% to about 90% by weight of the emulsion. In certain embodiments, water is present in an amount from about 30% to about 80% by weight of the emulsion. In certain embodiments, water is present in an amount of about 30%, about 32%, about 34%, about 36%, about 38%, about 40%, about 42%, about 44%, about 46%, about 48%, about 50%, about 52%, about 54%, about 56%, about 58%, about 60%, about 62%, about 64%, about 66%, about 68%, about 70%, about 72%, about 74%, about 76%, about 78%, or about 80% by weight of the emulsion. In certain embodiments, the water is purified water.

In certain embodiments, the invention relates to any one of the aforementioned emulsions, further comprising a buffer. In certain embodiments, the buffer is present in an amount from about 0.5% to about 1.5% by weight of the emulsion. In certain embodiments, the buffer is present in an amount from about 0.6% to about 1.4% by weight of the emulsion. In certain embodiments, the buffer is present about 0.6%, about 0.7%, about 0.8%, about 0.9%, about 1.0%, about 1.1%, about 1.2%, about 1.3%, or about 1.4% by weight of the emulsion.

In certain embodiments, the invention relates to any one of the aforementioned emulsions, further comprising a buffer. In certain embodiments, the buffer is present in an amount from about 0.50% to about 1.50% by weight of the emulsion. In certain embodiments, the buffer is present in an amount from about 0.60% to about 1.40% by weight of the emulsion. In certain embodiments, the buffer is present about 0.60%, about 0.70%, about 0.80%, about 0.90%, about 1.00%, about 1.10%, about 1.20%, about 1.30%, or about 1.40% by weight of the emulsion.

In certain embodiments, the invention relates to any one of the aforementioned emulsions, further comprising a pH adjuster. In certain embodiments, the pH adjuster is present in an amount from about 0.01% to about 0.3% by weight of the emulsion. In certain embodiments, the pH adjuster is present in an amount from about 0.02% to about 0.25% by weight of the emulsion. In certain embodiments, the pH adjuster is present about 0.02%, about 0.03%, about 0.04%, about 0.05%, about 0.06%, about 0.07%, about 0.08%, about 0.09%, about 0.10%, about 0.12%, about 0.14%, about 0.16%, about 0.18%, about 0.20%, about 0.22%, or about 0.24% by weight of the emulsion.

In certain embodiments, the invention relates to any one of the aforementioned emulsions, further comprising a pH adjuster. In certain embodiments, the pH adjuster is present in an amount from about 0.01% to about 0.30% by weight of the emulsion. In certain embodiments, the pH adjuster is present in an amount from about 0.02% to about 0.25% by weight of the emulsion. In certain embodiments, the pH adjuster is present about 0.02%, about 0.03%, about 0.04%, about 0.05%, about 0.06%, about 0.07%, about 0.08%, about 0.09%, about 0.10%, about 0.12%, about 0.14%, about 0.16%, about 0.18%, about 0.20%, about 0.22%, or about 0.24% by weight of the emulsion.

Exemplary Active Agents

As outlined above, in certain embodiments, the invention relates to an emulsion comprising an active agent. In certain embodiments, the active agent is reactive. The components described below may be present in any one of the aforementioned emulsions.

In certain embodiments, the invention relates to any one of the aforementioned emulsions, wherein the active agent is present in an amount from about 0.001% to about 15% by weight of the emulsion. In certain embodiments, the invention relates to any one of the aforementioned emulsions, wherein the active agent is present in an amount from about 0.05% to about 13% by weight of the emulsion. In certain embodiments, the invention relates to any one of the aforementioned emulsions, wherein the active agent is present in about 0.06%, about 0.08%, about 0.10%, about 0.12%, about 0.14%, about 0.16%, about 0.18%, about 0.2%, about 0.4%, about 0.5%, about 1.0%, about 1.5%, about 2.0%, about 2.5%, about 3.0%, about 3.5%, about 4.0%, about 4.5%, about 5%, about 6%, about 7%, about 8%, about 9%, about 10%, about 11%, about 12%, or about 13% by weight of the emulsion.

In certain embodiments, the invention relates to any one of the aforementioned emulsions, wherein the active agent is present in an amount from about 0.001% to about 15.00% by weight of the emulsion. In certain embodiments, the invention relates to any one of the aforementioned emulsions, wherein the active agent is present in an amount from about 0.05% to about 13.00% by weight of the emulsion. In certain embodiments, the invention relates to any one of the aforementioned emulsions, wherein the active agent is present in about 0.06%, about 0.08%, about 0.10%, about 0.12%, about 0.14%, about 0.16%, about 0.18%, about 0.20%, about 0.40%, about 0.50%, about 1.00%, about 1.50%, about 2.00%, about 2.50%, about 3.00%, about 3.50%, about 4.00%, about 4.50%, about 5.00%, about 6.00%, about 7.00%, about 8.00%, about 9.00%, about 10.00%, about 11.00%, about 12.00%, or about 13.00% by weight of the emulsion.

In certain embodiments, the invention relates to any one of the aforementioned emulsions, wherein the active agent is selected from the group consisting of retinoids such as retinol, tazarotene, adapalene, retinoic acid (e.g., tretinoin) and its derivatives (e.g., cis and trans, esters), and combinations/mixtures thereof.

In certain embodiments, the invention relates to any one of the aforementioned emulsions, wherein the active agent comprises tazarotene.

In certain embodiments, the invention relates to any one of the aforementioned emulsions, wherein the active agent comprises adapalene.

In certain embodiments, the invention relates to any one of the aforementioned emulsions, wherein the active agent comprises tretinoin. In certain embodiments, the tretinoin is present in an amount from about 0.05% to about 1.00% by weight of the emulsion. In certain embodiments, the tretinoin is present in an amount from about 0.07% to about 0.13% by weight of the emulsion. In certain embodiments, the tretinoin is present in about 0.07%, about 0.08%, about 0.09%, about 0.10%, about 0.11%, about 0.12%, or about 0.13% by weight of the emulsion.

In certain embodiments, the invention relates to any one of the aforementioned emulsions, wherein the active agent is a peroxide.

In certain embodiments, the invention relates to any one of the aforementioned emulsions, wherein the active agent is selected from the group consisting of benzoyl peroxide, alfa terpineol, octopirox, erythromycin, zinc, tetracyclin, triclosan, azelaic acid and its derivatives, phenoxy ethanol and phenoxy propanol, ethyl acetate, clindamycin and meclocycline, sebostats, alpha and beta hydroxy acids, and bile salts.

In certain embodiments, the invention relates to any one of the aforementioned emulsions, wherein the active agent comprises benzoyl peroxide. In certain embodiments, the benzoyl peroxide is present in an amount from about 0.05% to about 15% by weight of the emulsion. In certain embodiments, the benzoyl peroxide is present in an amount from about 7% to about 13% by weight of the emulsion. In certain embodiments, the benzoyl peroxide is present in about 7%, about 8%, about 9%, about 10%, about 11%, about 12%, or about 13% by weight of the emulsion.

In certain embodiments, the invention relates to any one of the aforementioned emulsions, wherein the active agent comprises benzoyl peroxide. In certain embodiments, the benzoyl peroxide is present in an amount from about 0.05% to about 15.00% by weight of the emulsion. In certain embodiments, the benzoyl peroxide is present in an amount from about 7.00% to about 13.00% by weight of the emulsion. In certain embodiments, the benzoyl peroxide is present in about 7.00%, about 8.00%, about 9.00%, about 10.00%, about 11.00%, about 12.00%, or about 13.00% by weight of the emulsion.

In certain embodiments, the invention relates to any one of the aforementioned emulsions, wherein the active agent comprises alfa terpineol. In certain embodiments, the alfa terpineol is present in an amount from about 1.5% to about 4.5% by weight of the emulsion. In certain embodiments, the alfa terpineol is present in an amount from about 1.8% to about 4.2% by weight of the emulsion. In certain embodiments, the alfa terpineol is present in about 1.8%, about 2.0%, about 2.2%, about 2.4%, about 2.6%, about 2.8%, about 3.0%, about 3.2%, about 3.4%, about 3.6%, about 3.8%, about 4.0%, or about 4.2% by weight of the emulsion.

In certain embodiments, the invention relates to any one of the aforementioned emulsions, wherein the active agent comprises alfa terpineol. In certain embodiments, the alfa terpineol is present in an amount from about 1.50% to about 4.50% by weight of the emulsion. In certain embodiments, the alfa terpineol is present in an amount from about 1.80% to about 4.20% by weight of the emulsion. In certain embodiments, the alfa terpineol is present in about 1.80%, about 2.00%, about 2.20%, about 2.40%, about 2.60%, about 2.80%, about 3.00%, about 3.20%, about 3.40%, about 3.60%, about 3.80%, about 4.00%, or about 4.20% by weight of the emulsion.

In certain embodiments, the invention relates to any one of the aforementioned emulsions, wherein the active agent is a vitamin. In certain embodiments, the vitamin is vitamin E (e.g., tocopherol) or a derivative thereof (e.g., tocopheryl acetate).

In certain embodiments, the invention relates to any one of the aforementioned emulsions, wherein the active agent is a quinone. In certain embodiments, the invention relates to any one of the aforementioned emulsions, wherein the active agent is hydroquinone.

### Exemplary Compositions of the Invention

In certain embodiments, the invention relates to a composition, comprising:

any one of the aforementioned emulsions; and

a propellant.

In certain embodiments, the invention relates to a composition, consisting essentially of:

any one of the aforementioned emulsions; and

a propellant.

In certain embodiments, the invention relates to a composition, consisting of:

any one of the aforementioned emulsions; and

a propellant.

In certain embodiments, the invention relates to a composition, comprising:

any one of the aforementioned emulsions;

a propellant; and

a purge gas.

In certain embodiments, the invention relates to a composition, consisting essentially of:

any one of the aforementioned emulsions;

a propellant; and

a purge gas.

In certain embodiments, the invention relates to a composition, consisting of:

any one of the aforementioned emulsions;

a propellant; and

a purge gas.

Exemplary Propellants

As outlined above, in certain embodiments, the invention relates to a composition comprising an emulsion, a propellant, and a purge gas. The propellants described below may be present in any one of the aforementioned compositions.

In certain embodiments, the invention relates to any one of the aforementioned compositions, wherein the propellant is selected from the group consisting of Z-1,2,3,3,3-pentafluoropropene (HFO-1225yeZ), E-1,2,3,3,3-pentafluoropropene (HFO-1225yeE), 1,1,3,3,3-pentafluoropropene (HFO-1225zc), 1,1,2,3,3-pentafluoropropene (HFO-1225yc), 2,3,3,3-tetrafluoropropene (HFO-1234yf), trans-1,3,3,3-tetrafluoroprop-1-ene (HFO-1234zeE or HBA-1), cis-1,3,3,3-tetrafluoropropene (HFO-1234zeZ), and 3,3,3-trifluoropropene (HFO-1243zf), and combinations/mixtures thereof.

In certain embodiments, the invention relates to any one of the aforementioned compositions, wherein the propellant is present in an amount from about 3% to about 20% by weight of the composition. In certain embodiments, the invention relates to any one of the aforementioned compositions, wherein the propellant is present in an amount from about 5% to about 18% by weight of the composition. In certain embodiments, the invention relates to any one of the aforementioned compositions, wherein the propellant is about 5%, about 6%, about 7%, about 8%, about 9%, about 10%, about 11%, about 12%, about 13%, about 14%, about 15%, about 16%, about 17%, or about 18% by weight of the composition.

Exemplary Purge Gases

As outlined above, in certain embodiments, the invention relates to a composition comprising an emulsion, a propellant, and a purge gas. The purge gases described below may be present in any one of the aforementioned compositions.

In certain embodiments, the invention relates to any one of the aforementioned compositions, wherein the purge gas is selected from the group consisting of nitrogen and argon. In certain embodiments, the invention relates to any one of the aforementioned compositions, wherein the purge gas is argon.

In certain embodiments, the invention relates to any one of the aforementioned compositions, wherein the purge gas is present in an amount from about 0.4% to about 6% by weight of the composition. In certain embodiments, the invention relates to any one of the aforementioned compositions, wherein the purge gas is present in an amount from about 0.8% to about 5% by weight of the composition. In certain embodiments, the invention relates to any one of the aforementioned compositions, wherein the purge gas is about 0.8%, about 1%, about 1.2%, about 1.4%, about 1.6%, about 1.8%, about 2%, about 2.2%, about 2.5%, about 2.6%, about 2.8%, about 3%, about 3.2%, about 3.4%, about 3.6%, about 3.8%, about 4%, about 4.2%, about 4.4%, about 4.6%, about 4.8% or about 5% by weight of the composition.

### Exemplary Properties of Emulsions and Compositions of the Invention

In certain embodiments, the invention relates to any one of the aforementioned emulsions or compositions that, upon application to the skin of an affected subject, is non-irritating.

In certain embodiments, the invention relates to any one of the aforementioned emulsions or compositions that, upon application to the skin of an affected subject, is well-tolerated.

In certain embodiments, the invention relates to any one of the aforementioned emulsions or compositions that, upon application to the skin of an affected subject, reduces inflammation.

In certain embodiments, the invention relates to any one of the aforementioned emulsions or compositions that, upon application to the skin of an affected subject, is non-cytotoxic.

In certain embodiments, the invention relates to any one of the aforementioned emulsions or compositions that, upon application to the skin of an affected subject, is weakly sensitizing. In certain embodiments, the invention relates to any one of the aforementioned emulsions or compositions that, upon application to the skin of an affected subject, is non-sensitizing.

In certain embodiments, the invention relates to any one of the aforementioned emulsions or compositions that, upon application to the skin of an affected subject, does not produce edema or erythema.

In certain embodiments, the invention relates to any one of the aforementioned emulsions or compositions that, upon application to the skin of an affected subject, moisturizes the skin.

In certain embodiments, the invention relates to any one of the aforementioned emulsions or compositions that, upon application to the skin of an affected subject, increases hydration of the skin.

In certain embodiments, the invention relates to any one of the aforementioned emulsions or compositions that, upon application to the skin of an affected subject, reduces transepidermal water loss.

In certain embodiments, the invention relates to any one of the aforementioned emulsions, wherein the emulsion has a viscosity of from about 75,000 cps to about 450,000 cps. In certain embodiments, the invention relates to any one of the aforementioned emulsions, wherein the emulsion has a viscosity of from about 100,000 cps to about 350,000 cps. In certain embodiments, the invention relates to any one of the aforementioned emulsions, wherein the emulsion has a viscosity of from about 150,000 cps to about 250,000 cps. In certain embodiments, the invention relates to any one of the aforementioned emulsions, wherein the emulsion has a viscosity of about 100,000 cps, about 125,000 cps, about 150,000 cps, about 175,000 cps, about 200,000 cps, about 225,000 cps, about 250,000 cps, about 275,000 cps, about 300,000 cps, about 325,000 cps, or about 350,000 cps.

In certain embodiments, the invention relates to any one of the aforementioned compositions that, upon expulsion from an aerosol container, forms a foam. In certain embodiments, the foam is temperature-stable. In certain embodiments, the foam is time-stable. In certain embodiments, the density of the foam is from about 0.05 to about 0.5 g/cm³. In certain embodiments, the invention relates to any one of the aforementioned compositions that is easily shaken in an aerosol container. In certain embodiments, the invention relates to any one of the aforementioned compositions that is easily dispensed from an aerosol container.

### Exemplary Emulsions and Compositions of the Invention for Particular Uses

In certain embodiments, the invention relates to any one of the aforementioned emulsions or compositions for use in the treatment of a skin disorder.

In certain embodiments, the invention relates to any one of the aforementioned emulsions or compositions for use in the treatment of a skin disorder, wherein the emulsion or composition is formulated for topical application once daily or twice daily.

### Exemplary Methods of Use

In certain embodiments, the invention relates to a method of treating a skin disorder, comprising the step of applying to an affected area of a subject in need thereof a therapeutically-effective amount of any one of the aforementioned emulsions or compositions.

In one embodiment, the present invention relates to any one of the above-mentioned methods, wherein the subject is human.

In one embodiment, the present invention relates to any one of the above-mentioned methods, wherein the composition is applied once daily.

In one embodiment, the present invention relates to any one of the above-mentioned methods, wherein the composition is applied twice daily.

### EXEMPLIFICATION

The invention now being generally described, it will be more readily understood by reference to the following examples which are included merely for purposes of illustration of certain aspects and embodiments of the present invention, and are not intended to limit the invention.

### Example 1

### Compositions and Method of Manufacture of Aerosol Foam Formulations

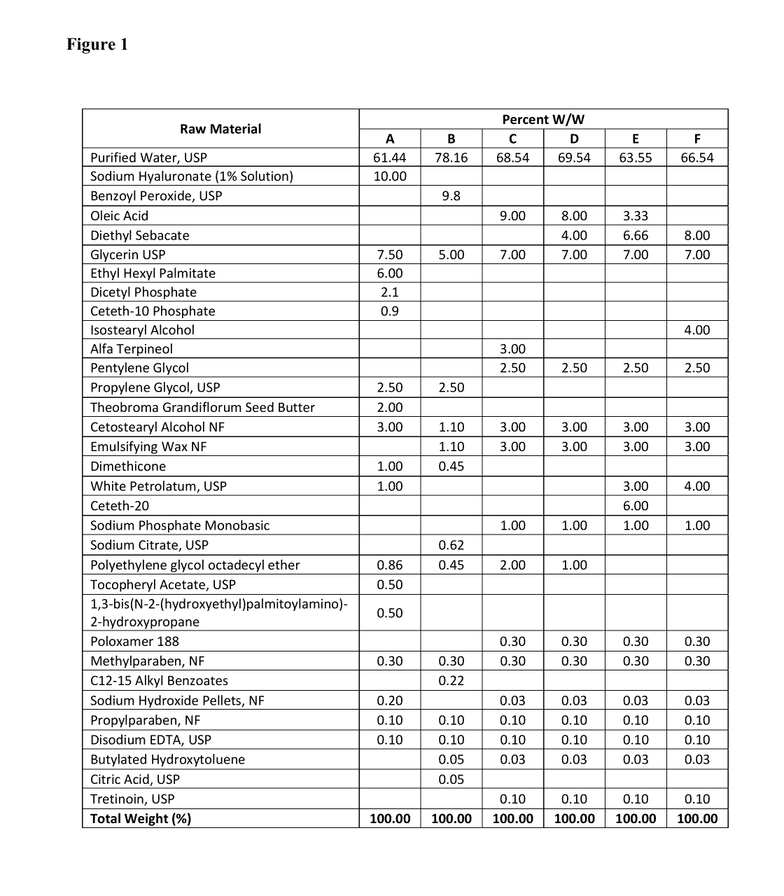

In one embodiment, the aerosol foam formulation is manufactured as outlined below. Various exemplary emulsion compositions are found in FIG. 1.

### Step 1: Oil Phase Preparation

1. Charge surfactants/co-surfactants and emollients into a Stainless Steel tank and heat to 75-80° C.

### Step 2: Aqueous Phase Preparation

1. Charge purified water (Part A) and humectants into a Stainless Steel tank and heat to 75-80° C.
2. Charge and dissolve preservatives and buffer salts while mixing.
3. Continue mixing until a clear solution is obtained while maintaining a temperature of 75-80° C.

### Step 3: Post-Emulsion Phase Preparation

1. Charge purified water (Part B) into stainless steel tank

2. Dissolve Sodium Hyaluronate while mixing

### Step 4: Final Emulsion Formation

1. Add Step 1 to Step 2 while high shear mixing at 75-80° C.
2. Cool the emulsion with an outside cold water jacket to 45-50° C. while high shear mixing.
3. Discontinue high shear mixing. Start low shear mixing and continue cooling with cold water jacket to form the vehicle emulsion.
4. When the temperature of the vehicle emulsion is 37° C., add Step 3 and continue mixing until uniform.
5. Cool to 27-32° C. Adjust to final pH with Sodium Hydroxide and final volume with DI water. Mix until uniform.

Following manufacturing of the Product Concentrate, the finished Product Concentrate may be filled into aerosol cans to be dispensed as a foam as outlined below.

1. Aerosol cans are cleaned with compressed air and vacuum.

2. Product Concentrate is filled into cans.

3. Valves are placed onto the cans.

4. Cans are crimped and hydrofluoroolefin propellant is charged.

5. The aerosol can valve and dip-tube is purged with argon gas.

Propellant concentrations range from about 5%-about 20% by weight of packaged product, argon concentrations range from about 0.8%-about 4.0% by weight of packaged product.

### Example 2

### Chemical Stability of Reactive Active Ingredients in the Presence of HFO Aerosol Propellants

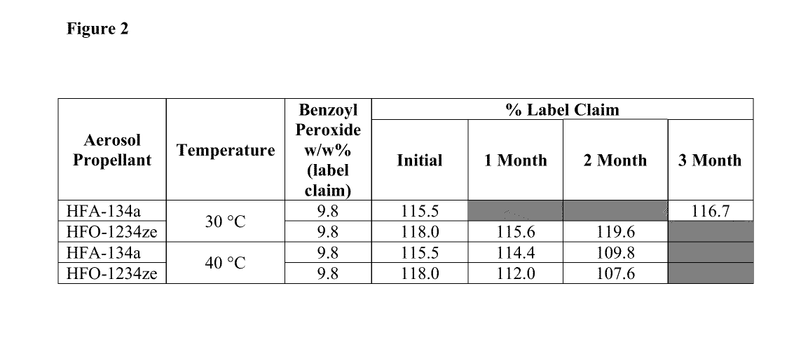

The composition B of Example 1 (FIG. 1) was tested for active ingredient chemical stability in the presence of either HFO or HFA propellants at 30° C. and 40° C. As can be seen from FIG. 2, even in the presence of the reactive aerosol propellant there is no substantive decrease in the chemical stability of the incorporated benzoyl peroxide.

### INCORPORATION BY REFERENCE

All of the U.S. patents and U.S. published patent applications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A composition, comprising:
an emulsion;
a propellant; and
a purge gas,
wherein
the emulsion comprises:
water,
an emulsifier or surfactant,
a moisturizer or emollient,
an antioxidant or preservative, and
an active agent; and
the propellant is selected from the group consisting of Z-1,2,3,3,3-pentafluoropropene, E-1,2,3,3,3-pentafluoropropene, 1,1,3,3,3-pentafluoropropene, 1,1,2,3,3-pentafluoropropene, 2,3,3,3-tetrafluoropropene, trans-1,3,3,3-tetrafluoroprop-1-ene, cis-1,3,3,3-tetrafluoropropene, and 3,3,3-trifluoropropene, and combinations/mixtures thereof.

2. The composition of claim 1, wherein the emulsion further comprises a pH adjuster.

3. The composition of claim 1, wherein the emulsion further comprises a buffer.

4. The composition of claim 1, wherein the emulsion comprises:
water, from about 24% to about 90% by weight of the emulsion;
sodium hyaluronate, from about 0.05% to about 1.5% by weight of the emulsion;
glycerin, from about 2.5% to about 10% by weight of the emulsion;
ethylhexyl palmitate, from about 3% to about 9% by weight of the emulsion;
cetostearyl alcohol, from about 0.5% to about 5% by weight of the emulsion;
dicetyl phosphate, from about 1% to about 3% by weight of the emulsion;
ceteth-10 phosphate, from about 0.5% to about 1.5% by weight of the emulsion;
propylene glycol, from about 1% to about 4% by weight of the emulsion;
theobroma grandiflorum seed butter, from about 1% to about 3% by weight of the emulsion;
dimethicone, from about 0.2% to about 2% by weight of the emulsion;
petrolatum, from about 0.5% to about 6% by weight of the emulsion;
polyethylene glycol octadecyl ether, from about 0.2% to about 3% by weight of the emulsion;
tocopheryl acetate, from about 0.2% to about 0.8% by weight of the emulsion;
1,3-bis(N-2-(hydroxyethyl)palmitoylamino)-2-hydroxypropane, from about 0.2% to about 0.8% by weight of the emulsion;
methylparaben, from about 0.01% to about 0.5% by weight of the emulsion;
sodium hydroxide, from about 0.01% to about 0.3% by weight of the emulsion;
propylparaben, from about 0.05% to about 0.2% by weight of the emulsion; and
disodium EDTA, from about 0.05% to about 0.2% by weight of the emulsion.

5. The composition of claim 1, wherein the emulsion comprises:
water, from about 24% to about 90% by weight of the emulsion;
benzoyl peroxide, from about 0.05% to about 15% by weight of the emulsion;
glycerin, from about 2.5% to about 10% by weight of the emulsion;
propylene glycol, from about 1% to about 4% by weight of the emulsion;
cetostearyl alcohol, from about 0.5% to about 5% by weight of the emulsion;
emulsifying wax, from about 0.5% to about 5% by weight of the emulsion;
dimethicone, from about 0.2% to about 2% by weight of the emulsion;
sodium citrate, from about 0.3% to about 0.9% by weight of the emulsion;
Polyethylene glycol octadecyl ether, from about 0.2% to about 3% by weight of the emulsion;
methylparaben, from about 0.01% to about 0.5% by weight of the emulsion;
C₁₂-C₁₅ alkyl benzoates, from about 0.1% to about 0.3% by weight of the emulsion;
propylparaben, from about 0.05% to about 0.2% by weight of the emulsion;
disodium EDTA, from about 0.05% to about 0.2% by weight of the emulsion;
butylated hydroxytoluene, from about 0.01% to about 0.08% by weight of the emulsion; and
citric acid, from about 0.02% to about 0.08% by weight of the emulsion.

6. The composition of claim 1, wherein the emulsion comprises:
water, from about 24% to about 90% by weight of the emulsion;
oleic acid, from about 1.5% to about 14% by weight of the emulsion;
glycerin, from about 2.5% to about 10% by weight of the emulsion;
alfa terpineol, from about 1.5% to about 4.5% by weight of the emulsion;
pentylene glycol, from about 1% to about 4% by weight of the emulsion;
cetostearyl alcohol, from about 0.5% to about 5% by weight of the emulsion;
emulsifying wax, from about 0.5% to about 5% by weight of the emulsion;
sodium phosphate monobasic, from about 0.5% to about 1.5% by weight of the emulsion;
Polyethylene glycol octadecyl ether, from about 0.2% to about 3% by weight of the emulsion;
Poloxamer 188, from about 0.01% to about 2% by weight of the emulsion;
methylparaben, from about 0.01% to about 0.5% by weight of the emulsion;
sodium hydroxide, from about 0.01% to about 0.3% by weight of the emulsion;
propylparaben, from about 0.05% to about 0.2% by weight of the emulsion;
disodium EDTA, from about 0.05% to about 0.2% by weight of the emulsion;
butylated hydroxytoluene, from about 0.01% to about 0.08% by weight of the emulsion; and
tretinoin, from about 0.05% to about 1% by weight of the emulsion.

7. The composition of claim 1, wherein the emulsion comprises:
water, from about 24% to about 90% by weight of the emulsion;
oleic acid, from about 1.5% to about 14% by weight of the emulsion;
diethyl sebacate, from about 2% to about 12% by weight of the emulsion;
glycerin, from about 2.5% to about 10% by weight of the emulsion;
pentylene glycol, from about 1% to about 4% by weight of the emulsion;
cetostearyl alcohol, from about 0.5% to about 5% by weight of the emulsion;
emulsifying wax, from about 0.5% to about 5% by weight of the emulsion;
sodium phosphate monobasic, from about 0.5% to about 1.5% by weight of the emulsion;
Polyethylene glycol octadecyl ether, from about 0.2% to about 3% by weight of the emulsion;
Poloxamer 188, from about 0.01% to about 2% by weight of the emulsion;
methylparaben, from about 0.01% to about 0.5% by weight of the emulsion;
sodium hydroxide, from about 0.01% to about 0.3% by weight of the emulsion;
propylparaben, from about 0.05% to about 0.2% by weight of the emulsion;
disodium EDTA, from about 0.05% to about 0.2% by weight of the emulsion;
butylated hydroxytoluene, from about 0.01% to about 0.08% by weight of the emulsion; and
tretinoin, from about 0.05% to about 1% by weight of the emulsion.

8. The composition of claim 1, wherein the emulsion comprises:
water, from about 24% to about 90% by weight of the emulsion;
oleic acid, from about 1.5% to about 14% by weight of the emulsion;
diethyl sebacate, from about 2% to about 12% by weight of the emulsion;
glycerin, from about 2.5% to about 10% by weight of the emulsion;
pentylene glycol, from about 1% to about 4% by weight of the emulsion;
cetostearyl alcohol, from about 0.5% to about 5% by weight of the emulsion;
emulsifying wax, from about 0.5% to about 5% by weight of the emulsion;
petrolatum, from about 0.5% to about 6% by weight of the emulsion;
Ceteth-20, from about 3% to about 9% by weight of the emulsion;
sodium phosphate monobasic, from about 0.5% to about 1.5% by weight of the emulsion;
Poloxamer 188, from about 0.01% to about 2% by weight of the emulsion;
methylparaben, from about 0.01% to about 0.5% by weight of the emulsion;
sodium hydroxide, from about 0.01% to about 0.3% by weight of the emulsion;
propylparaben, from about 0.05% to about 0.2% by weight of the emulsion;
disodium EDTA, from about 0.05% to about 0.2% by weight of the emulsion;
butylated hydroxytoluene, from about 0.01% to about 0.08% by weight of the emulsion; and
tretinoin, from about 0.05% to about 1% by weight of the emulsion.

9. The composition of claim 1, wherein the emulsion comprises:
water, from about 24% to about 90% by weight of the emulsion;
diethyl sebacate, from about 2% to about 12% by weight of the emulsion;
glycerin, from about 2.5% to about 10% by weight of the emulsion;
isostearyl alcohol, from about 2% to about 6% by weight of the emulsion;
pentylene glycol, from about 1% to about 4% by weight of the emulsion;
cetostearyl alcohol, from about 0.5% to about 5% by weight of the emulsion;
emulsifying wax, from about 0.5% to about 5% by weight of the emulsion;
petrolatum, from about 0.5% to about 6% by weight of the emulsion;
sodium phosphate monobasic, from about 0.5% to about 1.5% by weight of the emulsion;
Poloxamer 188, from about 0.01% to about 2% by weight of the emulsion;
methylparaben, from about 0.01% to about 0.5% by weight of the emulsion;
sodium hydroxide, from about 0.01% to about 0.3% by weight of the emulsion;
propylparaben, from about 0.05% to about 0.2% by weight of the emulsion;
disodium EDTA, from about 0.05% to about 0.2% by weight of the emulsion;
butylated hydroxytoluene, from about 0.01% to about 0.08% by weight of the emulsion; and
tretinoin, from about 0.05% to about 1% by weight of the emulsion.

10. The composition of claim 1, wherein the emulsifier or surfactant is selected from the group consisting of: polysorbate 20, polysorbate 40, polysorbate 60, polysorbate 80, steareth-10, sodium dodecyl sulfate, lauryl dimethyl amine oxide, cetyltrimethylammonium bromide, polyethoxylated alcohols, polyoxyethylene sorbitan, octoxynol, N,N-dimethyldodecylamine-N-oxide, hexadecyltrimethylammonium bromide, polyoxyl 10 lauryl ether, sodium deoxycholate, sodium cholate, polyoxyl castor oil, nonylphenol ethoxylate, cyclodextrins, lecithin, dimethicone copolyol, lauramide DEA, cocamide DEA, cocamide MEA, oleyl betaine, cocamidopropyl betaine, cocamidopropyl phosphatidyl PG-dimonium chloride, dicetyl phosphate, ceteareth-10 phosphate, methylbenzethonium chloride, behentrimonium methosulfate-cetearyl alcohol, non-ionic emulsifiers like emulsifying wax, polyoxyethylene oleyl ether, PEG-40 stearate, ceteareth-12, ceteareth-20, ceteareth-30, ceteareth alcohol, Ceteth-20, oleic acid, oleyl alcohol, glyceryl stearate, PEG-100 stearate, glyceryl stearate and PEG-100 stearate, steareth-2, steareth-20, stearic acid, cholesterol, ceramide 2, ceramide 3, stearamidopropyl dimethylamine, behentrimonium methosulfate, cetostearyl alcohol, dicetyl phosphate, ceteth-10 phosphate, polyethylene glycol octadecyl ether, Poloxamer 188, and combinations/mixtures thereof.

11. The composition of claim 1, wherein the emulsifier or surfactant is present in an amount from about 1% to about 25% by weight of the emulsion.

12. The composition of claim 1, wherein the moisturizer or emollient is selected from the group consisting of petrolatum, glycerol (glycerin), sodium hyaluronate, 2-ethylhexyl palmitate, dimethicone, C₁₂-C₁₅ alkyl benzoates, propylene glycol, Theobroma grandiflorum seed butter, diethyl sebacate, isostearyl alcohol, pentylene glycol, 1,3-bis(N-2-(hydroxyethyl)palmitoylamino)-2-hydroxypropane, and combinations/mixtures thereof.

13. The composition of claim 1, wherein the moisturizer or emollient is present in an amount from about 4% to about 30% by weight of the emulsion.

14. The composition of claim 1, wherein the antioxidant or preservative is selected from the group consisting of imidazolidinyl urea, diazolidinyl urea, phenoxyethanol, sodium methyl paraben, methylparaben, ethylparaben, propylparaben, potassium sorbate, sodium benzoate, sorbic acid, benzoic acid, formaldehyde, citric acid, sodium citrate, chlorine dioxide, benzalkonium chloride, benzethonium chloride, cetrimide, dequalinium chloride, cetylpyridinium chloride, phenylmercuric nitrate, phenylmercuric acetate, thimerosal, piroctone olamine, Vitis vinifera seed oil, chlorobutanol, dichlorobenzyl alcohol, phenylethyl alcohol, benzyl alcohol, ascorbic acid, sodium bisulfite, butylated hydroxytoluene, butylated hydroxyanisole, α-tocopherol, tocopheryl acetate, sodium ascorbate/ascorbic acid, ascorbyl palmitate, propyl gallate, disodium EDTA, citric acid, and sodium citrate, and combinations/mixtures thereof.

15. The composition of claim 1, wherein the antioxidant or preservative is present in an amount from about 0.25% to about 2% by weight of the emulsion.

16. The composition of claim 1, wherein water is present in an amount about 24% to about 90% by weight of the emulsion.

17. The composition of claim 1, wherein the active agent is present in an amount from about 0.001% to about 15% by weight of the emulsion.

18. The composition of claim 1, wherein the active agent is a retinoid.

19. The composition of claim 1, wherein the active agent is a peroxide.

20. The composition of claim 1, wherein the active agent is selected from the group consisting of benzoyl peroxide, alfa terpineol, octopirox, erythromycin, zinc, tetracyclin, triclosan, azelaic acid and its derivatives, phenoxy ethanol and phenoxy propanol, ethyl acetate, clindamycin and meclocycline, sebostats, alpha and beta hydroxy acids, and bile salts.

21. The composition of claim 1, wherein the active agent is a vitamin.

22. The composition of claim 1, wherein the active agent is a quinone.

23. A method of treating a skin disorder, comprising the step of applying to an affected area of a subject in need thereof a therapeutically-effective amount of a composition of claim 1.
